(19) United States
(12) Patent Application Publication
(10) Pub. No.: US 2011/0144371 A1
(43) Pub. Date: Jun. 16, 2011

(54) PROCESS FOR PURIFYING DIALKYL CARBONATES

(75) Inventors: Pieter Ooms, Krefeld (DE); Friedhelm Risse, Koln (DE); Andre Düx, Bruhl (DE); Carsten Buchaly, Dusseldorf (DE); Thomas Pancur, Altenholz (DE); Arthur Susanto, Koln (DE); Georg Ronge, Dusseldorf (DE); Johan Vanden Eynde, Zwijnaarde (BE); Wim Wuytack, Zele (BE)

(73) Assignee: Bayer MaterialScience AG, Leverkusen (DE)

(21) Appl. No.: 12/944,943

(22) Filed: Nov. 12, 2010

(30) Foreign Application Priority Data
Nov. 14, 2009 (DE) 102009053370.2

(51) Int. Cl. C07C 68/08

(52) U.S. Cl. 558/277

(57) ABSTRACT

The present invention relates to a special process for purifying dialkyl carbonates. In particular, the present invention relates to a continuous process for purifying a dialkyl carbonate/alcohol-mixture in the preparation of lower dialkyl carbonate by catalysed transesterification of a cyclic alkylene carbonate (e.g. ethylene carbonate or propylene carbonate) with lower alcohols. To optimize the economics and energy efficiency of the process, an apparatus for intermediate heating of the internal liquid stream is used.

### BACKGROUND OF THE INVENTION

1. Field of the Invention

The field of the present invention relates to a special process for purifying dialkyl carbonates. In particular, the present invention relates to a continuous process for purifying a dialkyl carbonate/alkyl alcohol-mixture in the preparation of dialkyl carbonate by catalysed transesterification of a cyclic alkylene carbonate (e.g. ethylene carbonate or propylene carbonate) with alkyl alcohols. To optimize the economics and energy efficiency of the process, an apparatus for intermediate heating of the internal liquid stream is used.

2. Background

The purification of dialkyl carbonate as a precursor of diaryl carbonate is of great importance because of the high purity required for the preparation of high quality polycarbonates by means of melt transesterification. Dialkyl carbonates prepared by transesterification of cyclic alkylene carbonate and alkyl alcohol can contain both high-boiling and low-boiling components and also catalyst residues as impurities. High-boiling components, often also referred to as high boilers, in the context of this purification process are those whose boiling point is above that of the dialkyl carbonate. Low-boiling components, often also referred to as low boilers, in the context of this purification process are those whose boiling point is below that of the dialkyl carbonate. All these impurities lead to a considerable deterioration in the quality of the diaryl carbonates to be prepared and also polycarbonates subsequently prepared therefrom and have to be removed by appropriate purification before further use of the dialkyl carbonates.

The preparation of dialkyl carbonates from cyclic alkylene carbonate and alkyl alcohol, which at the same time forms alkylene glycol as by-product, is known and has been described many times. U.S. Pat. No. 6,930,195 B, for example, has described this catalysed transesterification reaction as a two-stage equilibrium reaction. In the first reaction stage, the cyclic alkylene carbonate reacts with alcohol to form hydroxyalkyl carbonate as intermediate. The intermediate is then reacted with an alkyl alcohol in the second reaction stage to form the products: dialkyl carbonate and alkylene glycol.

The use of a reactive distillation column (hereinafter also referred to as transesterification column), which has been described, inter alia, in EP 530 615 A, EP 569 812 A and EP 1 086 940 A, has been found to be particularly advantageous for the industrial implementation of the dialkyl carbonate production process. In EP 569 812 A, the cyclic alkylene carbonate is introduced continuously into the upper part of the transesterification column and the dialkyl carbonate-containing alkyl alcohol is introduced continuously into the middle or lower part of the transesterification column. In addition, virtually pure alkyl alcohol is introduced below the point of introduction of the dialkyl carbonate-containing alkyl alcohol. The high boiler mixture, which contains the alkylene glycol produced as by-product, is taken off continuously at the bottom of the transesterification column. The low boiler mixture, which contains the dialkyl carbonate produced, is taken off as dialkyl carbonate/alkyl alcohol mixture at the top of the transesterification column and is subjected to a further purification step.

In the present application “virtually pure” includes mixtures of ≧99% of main component.

The distillation column for purifying the dialkyl carbonate/alkyl alcohol mixture is operated at a higher pressure so that a further dialkyl carbonate/alkyl alcohol mixture having a lower dialkyl carbonate content can be taken off at the top of the column. The dialkyl carbonate as main product is obtained at the bottom of this purification column.

Many factors play an important role in the development of an economically attractive production process for dialkyl carbonates. Most literature sources are concerned with the reaction parameters such as conversion, selectivity or product purity. The energy efficiency of the process is addressed more rarely (e.g. EP 569 812 A, JP 2003-104937, WO 2007/096340, WO 2007/096343), although these factors make a not inconsiderable contribution to the economic attractiveness of the process. For this reason, measures to increase the energy efficiency of the process are introduced in the present invention.

In EP 569 812 A, the energy consumption in the preparation of dialkyl carbonate is reduced by many internal streams in the process not being condensed but being conveyed as gaseous streams.

WO 2007/096340 describes a process in which alkylene carbonate is produced from alkylene oxide and CO₂ and the alkylene carbonate is subsequently reacted with alkyl alcohol to form dialkyl carbonate and alkylene glycol, with the mixture formed in the second step, which contains dialkyl carbonate and alkylene glycol, being purified. The reaction to form the alkylene carbonate is exothermic and the corresponding alkylene carbonate product stream is used to heat the dialkyl carbonate/alkylene glycol product stream in the purification.

In WO 2007/096343, the mixture of dialkyl carbonate and alkyl alcohol formed in a transesterification column from alkylene carbonate and alkyl alcohol is purified by means of extractive distillation, with alkylene carbonate serving as extractant. After the dialkyl carbonate has been separated from the extractant by distillation, the alkyl alcohol fed to the transesterification column is heated by means of the hot bottom product from this column, which contains the extractant.

In JP 2003-104937, various process variants for working up an ethylene carbonate/ethylene glycol mixture and providing the purified ethylene carbonate for the process for preparing dimethyl carbonate are also examined from the point of view of energy consumption.

It is found that the distillation of the low-boiling product stream coming from the transesterification column can be carried out only with a low energy efficiency and with a high outlay in terms of apparatus according to the processes of the prior art. An apparatus for reducing the energy input has not been known hitherto.

There was therefore a need to provide a process for purifying dialkyl carbonates, which does not have the abovementioned disadvantages and in which energy integration is possible in a more efficient way compared to the abovementioned known processes or improved energy integration can be achieved. Furthermore, there was a need for a process for purifying dialkyl carbonates, in which intermediate-boiling secondary components present as impurities can also be removed if necessary in an energetically favourable way which is very simple in terms of apparatus. Here, intermediate-boiling secondary components are those whose boiling point is between the boiling point of the alkyl alcohol and that of the dialkyl carbonate.

It was therefore an object of the invention to provide a process for purifying dialkyl carbonates, which has a reduced energy consumption compared to known processes.

### DETAILED DESCRIPTION OF THE PREFERRED EMBODIMENTS

It has now surprisingly been found that the purification of dialkyl carbonates by distillation can also be carried out with a low outlay in terms of apparatus and low energy consumption when the heat arising in the distillation and/or the subsequent process for preparing diaryl carbonate is utilized to provide energy.

In particular, the heat energy requirement at the temperature level T_{BV}, which is required for operation of the bottom vaporizer, can be reduced particularly simply and advantageously by the additional use of a technical apparatus for intermediate heating in the distillation column. The energy input in this intermediate heater comes predominantly from the subsequent process for preparing diaryl carbonate. Due to the lower temperature of the internal stream compared to the temperature at the bottom of the column, heat energy at the temperature level T_{I} where T_{I}<T_{BV} can be used for the intermediate heating. This concept leads to an overall reduction in the consumption of heat energy at a temperature level greater than or equal to T_{BV} because now the heat energy at a temperature level below T_{BV} obtained in other chemical production processes, e.g. in a condensation or in the cooling of a stream, can be utilized gainfully and the amount of generally costly heat energy at a temperature level greater than or equal to T_{BV} can be reduced.

The heat energy obtained at a temperature level T_{I} in other chemical production processes by condensation or cooling of a stream can be supplied either directly or indirectly to the intermediate heater. In the case of direct supply, the stream which is to be condensed or cooled in the other chemical production processes heats, by means of the intermediate heater, the internal stream in the distillation column for purifying the dialkyl carbonate. In the case of indirect supply, the stream to be condensed or cooled heats the internal stream in the column via one or more heat transfer media. Possible heat transfer media are gases, vapours or liquids, preferably gaseous or liquid industrial heat transfer media such as water, heat transfer media based on mineral oil or synthetic heat transfer media (e.g. Diphyl™, Marlotherm®). Particularly preferred heat transfer media are water or steam.

Accordingly, a process is provided for purifying dialkyl carbonates in at least one distillation column containing at least one enrichment section in the upper part of the column and at least one stripping section in the lower part of the column, characterized in that, in the distillation column for working up the dialkyl carbonate/alkyl alcohol mixture taken off at the top of the transesterification column, a technical apparatus for heating the internal liquid stream in the column is used, with the energy used being partly or entirely recovered from another chemical production process. This technical apparatus is preferably positioned in the stripping section of the column, particularly advantageously in the upper half of the stripping section of the column.

In the dialkyl carbonate purification column, the consumption of heat energy at the temperature level of T_{BV} in the bottom vaporizer can be reduced by intermediate heating in the stripping section of the column, particularly advantageously in the upper half of the stripping section of the column. The required amount of heat energy at the temperature level T_{I} for the intermediate heater can be obtained from another chemical production process. This is preferably the subsequent preparation of diaryl carbonate, with the required amount of heat energy preferably being able to be obtained from the heat of condensation arising in the intermediate condensation within the first reaction column of the preparation of diaryl carbonate and/or in the condensation at the top of the further reaction columns and/or further distillation columns of the preparation of diaryl carbonate and also the condensate system.

As a result of the reduction in the consumption of heat energy at the temperature level T_{BV} while simultaneously maintaining the high product quality, the process provides a significant economic advantage.

Dialkyl carbonates purified by the processes described herein are preferably all those of the general formula (I)

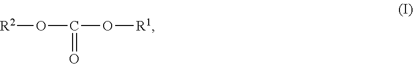

where R¹ and R² are each, independently of one another, linear or branched, optionally substituted C₁-C₃₄-alkyl, preferably C₁-C₆-alkyl, particularly preferably C₁-C₄-alkyl. R¹ and R² can be identical or different. R¹ and R² are preferably identical.

For the purposes of this description, C₁-C₄-alkyl is, for example, methyl, ethyl, n-propyl, isopropyl, n-butyl, sec-butyl, tert-butyl, C₁-C₆-alkyl can also be, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl, C₁-C₃₄-alkyl can also be, for example, n-heptyl and n-octyl, pinacyl, adamantyl, the isomeric menthyls, n-nonyl, n-decyl, n-dodecyl, n-tridecyl, n-tetradecyl, n-hexadecyl or n-octadecyl. The same applies to the corresponding alkyl radical in, for example, aralkyl or alkylaryl radicals. Alkylene radicals in the corresponding hydroxyalkyl or aralkyl or alkylaryl radicals are, for example, the alkylene radicals corresponding to the above alkyl radicals.

The listings above are by way of example and do not constitute a limitation.

Preferred dialkyl carbonates are dimethyl carbonate, diethyl carbonate, di(n-propyl)carbonate, di(isopropyl)carbonate, di(n-butyl)carbonate, di(sec-butyl)carbonate, di(tert-butyl)carbonate or dihexyl carbonate. Particular preference is given to dimethyl carbonate or diethyl carbonate. Very particular preference is given to dimethyl carbonate.

The dialkyl carbonates are preferably prepared from cyclic alkylene carbonates having the formula (II):

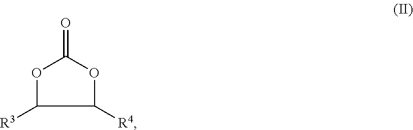

where, in the formula, R³ and R⁴ are each, independently of one another, hydrogen, substituted or unsubstituted C₁-C₄-alkyl, substituted or unsubstituted C₂-C₄-alkenyl or substituted or unsubstituted C₆-C₁₂-aryl and R³ and R⁴ together with the two carbon atoms of the three-membered ring can form a saturated carbocyclic ring having 5-8 ring atoms.

The cyclic alkylene carbonates are reacted with alcohols of the formula

R⁵—OH

where R⁵ is a straight-chain or branched C₁-C₄-alkyl.

Transesterification catalysts used for producing the dialkyl carbonates are known to those skilled in the art, for example hydrides, oxides, hydroxides, alkoxides, amides or salts of alkali metals such as lithium, sodium, potassium, rubidium and caesium, preferably of lithium, sodium and potassium, particularly preferably of sodium and potassium (U.S. Pat. No. 3,642,858 A, U.S. Pat. No. 3,803,201 A, EP 1 082 A). When alkoxides are used, these can also be formed in situ by use of the elemental alkali metals and the alcohol to be reacted. Salts of the alkali metals can be those of organic or inorganic acids, e.g. of acetic acid, propionic acid, butyric acid, benzoic acid, stearic acid, carbonic acid (carbonates or hydrogencarbonates), of hydrochloric acid, hydrobromic acid or hydroiodic acid, nitric acid, sulphuric acid, hydrofluoric acid, phosphoric acid, hydrocyanic acid, thiocyanic acid, boric acid, stannic acid, C₁-C₄-stannonic acids or antimonic acids. As compounds of the alkali metals, preference is given to the oxides, hydroxides, alkoxides, acetates, propionates, benzoates, carbonates and hydrogencarbonates, and particular preference is given to using hydroxides, alkoxides, acetates, benzoates or carbonates. Such alkali metal compounds (if appropriate formed in situ from the free alkali metals) are used in amounts of from 0.001 to 2% by weight, preferably from 0.003 to 1.0% by weight, particularly preferably from 0.005 to 1.0% by weight, based on the reaction mixture to be reacted.

It is possible to add, if appropriate, complexing substances to such alkali metal compounds. Mention may be made of, for example, crown ethers such as dibenzo-18-crown-6, polyethylene glycols or bicyclic nitrogen-containing cryptands.

Such complexing agents are used in amounts of from 0.1 to 200 mol %, preferably from 1 to 100 mol %, based on the alkali metal compound.

Further suitable catalysts for the preparation of dialkyl carbonates are thallium(I) and thallium(III) compounds such as the oxides, hydroxides, carbonates, acetates, bromides, chlorides, fluorides, formates, nitrates, cyanates, stearates, naphthenates, benzoates, cyclohexylphosphonates, hexahydrobenzoates, cyclopentadienylthallium, thallium methoxide, thallium ethoxide, preferably TI(I) oxide, TI(I) hydroxide, TI(I) carbonate, TI(I) acetate, TI(111) acetate, TI(I) fluoride, TI(I) formate, TI(I) nitrate, TI(I) napthenate and TI(I) methoxide (EP 1 083). The amounts of thallium catalyst are not particularly critical. They are generally 0.0001-10% by weight, preferably 0.001-1% by weight, based on the total reaction mixture. Furthermore, nitrogen-containing bases can be used as catalysts in the production process (U.S. Pat. No. 4,062,884). Mention may be made by way of example of secondary or tertiary amines such as triethylamine, tributylamine, methyldibenzylamine, dimethylcyclohexylamine, etc.

The amounts of nitrogen-containing bases used are from 0.01 to 10% by weight, preferably from 0.1 to 5% by weight, particularly preferably from 0.1 to 1% by weight, based on the total reaction mixture. Compounds from the group of phosphines, stibines, arsines or divalent sulphur and selenium compounds and also onium salts thereof can also be used as catalysts (EP 180 387, U.S. Pat. No. 4,734,519).

The following may be mentioned by way of example: tributylphosphine, triphenylphosphine, diphenylphosphine, 1,3-bis(diphenylphosphino)propane, triphenylarsine, trimethylarsine, tributylarsine, 1,2-bis(diphenylarsino)ethane, triphenylantimony, diphenyl sulphide, diphenyl disulphide, diphenyl selenide, tetraphenylphosphonium halide (Cl, Br, I), tetraphenylarsonium halide (Cl, Br, I), triphenylsulphonium halide (Cl, Br), etc.

The preferred use amounts of this group of catalysts are in the range from 0.1 to 10% by weight, preferably from 0.1 to 5% by weight, particularly preferably in the range from 0.1 to 2% by weight, based on the total reaction mixture.

Furthermore, compounds of tin, titanium or zirconium can be used as catalysts (U.S. Pat. No. 4,661,609 A). Examples of such systems are butylstannonic acid, tin methoxide, dimethyltin, dibutyltin oxide, dibutyltin dilaurate, tributyltin hydride, tributyltin chloride, tin(II)ethylhexanoate, zirconium alkoxides (methyl, ethyl, butyl), zirconium(IV) halides (F, Cl, Br, I), zirconium nitrates, zirconium acetylacetonate, titanium alkoxides (methyl, ethyl, isopropyl), titanium acetate, titanium acetylacetonate, etc.

The preferred amounts of these catalysts are from 0.1 to 10% by weight, preferably from 0.1 to 5% by weight, based on the total mixture.

Furthermore, bifunctional catalysts of the formula (III)

[AₐX_{b}]ₘ.[B_{c}Y_{d}]ₙ (III)

can be used in the production process. In these bifunctional catalysts, the molar ratio of the two components in square brackets is indicated by the indices m and n. These indices can, independently of one another, assume values of 0.001-1, preferably 0.01-1, particularly preferably 0.05-1 and very particularly preferably 0.1-1. The compounds within these square brackets are uncharged salts composed of a cation and an anion. The indices a and b are, independently of one another, integers of 1-5; the indices c and d are, independently of one another, integers of 1-3, with the valencies of the cations and anions having to meet the requirements for formation of such uncharged salts. Furthermore, in (III), A is the cation of a metal belonging to the third period and group IIa, the fourth period and group IIa, IVa-VIIIa, Ib or IIb, the fifth period and group IIa, IVa-VIIa or IVb or the sixth period and groups IIa-VIa of the Periodic Table of the Elements in the short period form.

The possible metals for the cation A can be taken by a person skilled in the art from the conventional presentation of the Periodic Table of the Elements (Mendeleev) in the short period form. A is preferably the cation of one of the metals Mg, Ca, Sr, Ba, Zn, Cu, Mn, Co, Ni, Fe, Cr, Mo, W, Ti, Zr, Sn, Hf, V and Ta, preferably the cation of one of the metals Mg, Ca, Zn, Co, Ni, Mn, Cu and Sn. Apart from the uncomplexed cations of the metals mentioned, cationic oxo complexes of the metals mentioned are also possible, for example titanyl TiO⁺⁺ and chromyl CrO₂⁺⁺.

The anion X associated with cation A is that of an inorganic or organic acid. Such an inorganic or organic acid can be monobasic or dibasic or tribasic. Such acids and their anions are known to those skilled in the art. Examples of anions of monobasic inorganic or organic acids are: fluoride, bromide, chloride, iodide, nitrate, the anion of an alkanecarboxylic acid having 1-18 carbon atoms and benzoate; examples of anions of dibasic inorganic or organic acids are: sulphate, oxalate, succinate, fumarate, maleate, phthalate and others; examples of tribasic inorganic or organic anions are: phosphate or citrate. Preferred anions X in the catalyst of the formula (III) are: fluoride, chloride, bromide, iodide, sulphate, nitrate, phosphate, formate, acetate, propionate, oxalate, butyrate, citrate, succinate, fumarate, maleate, benzoate, phthalate, decanoate, stearate, palmitate and laurate. Particularly preferred anions X are: chloride, bromide, iodide, acetate, laurate, stearate, palmitate, decanoate, nitrate and sulphate.

Possible cations B in the catalysts of the formula (III) are cations from the group consisting of alkali metal or alkaline earth metal cations, quaternary ammonium, phosphonium, arsonium or stibonium cations and ternary sulphonium cations.

As alkali metal/alkaline earth metal cations, mention may be made of: the lithium, sodium, potassium, rubidium, caseium, magnesium, calcium, strontium and barium cations, preferably the alkali metal cations mentioned, particularly preferably the sodium cation and the potassium cation.

Preferred cations B are those of the formula (IV)

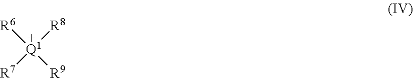

where:
Q¹ is N, P, As or Sb and
R⁶, R⁷, R⁸ and R⁹ are each, independently of one another, straight-chain or branched C₁-C₁₈-alkyl or C₇-C₁₂-aralkyl and one of the radicals R⁶-R⁹ can also be C₆-C₁₂-aryl. B is particularly preferably a cation of the formula (V)

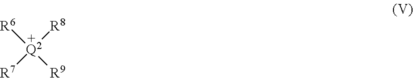

where:
Q² is N or P, preferably N.

In the formulae (IV) and (V), the radicals R⁶, R⁷, R⁸ and R⁹ are very particularly preferably replaced by radicals R¹⁶, R¹⁷, R¹⁸ and R¹⁹ which are each, independently of one another, straight-chain or branched C₁-C₁₈-alkyl or C₇-C₈-aralkyl and one of the radicals R¹⁶ to R¹⁹ can also be phenyl. The radicals R¹⁶, R¹⁷, R¹⁸ and R¹⁹ are even more particularly preferably replaced by radicals R²⁶, R²⁷, R28 and R²⁹ which are each, independently of one another, C₁-C₈-alkyl or benzyl and one of the radicals R²⁶ to R²⁹ can also be phenyl.

Straight-chain or branched C₁-C₁₈-alkyl is, for example, methyl, ethyl, propyl, isopropyl, butyl, isobutyl, hexyl, octyl, dodecyl, hexadecyl or octadecyl. Preferred alkyl has 1-12 carbon atoms, and particularly preferred alkyl has 1-8 carbon atoms.

C₇-C₁₂-Aralkyl is, for example, benzyl, phenylethyl, phenylpropyl, naphthylmethyl or naphthylethyl; preferred aralkyl is benzyl or phenylethyl, and very particularly preferred aralkyl is benzyl.

C₆-C₁₂-Aryl is, for example, phenyl, naphthyl or biphenylyl, preferably phenyl.

The anion Y in the catalyst of the formula (III) is a halide ion such as fluoride, chloride, bromide or iodide, preferably bromide or iodide, particularly preferably iodide. However, it can also be another one of the anions mentioned under X when in the specific case the anion X is bromide or iodide.

The bifunctional catalyst of the formula (III) is used in an amount of 0.005-5% by weight, preferably 0.01-3% by weight, particularly preferably 0.01-1% by weight, based on the total transesterification mixture.

Such catalysts can be introduced as homogeneous solutions at the top of the column, with alkylene carbonate, alkylene glycol, alcohol or dialkyl carbonate, i.e. solvents present in the system, being employed as solvents. It is of course also possible to use insoluble transesterification catalysts which are arranged on the intermediate trays or inside the packing elements. In such a case, introduction of a dissolved catalyst via (2) can be omitted. Suitable heterogeneous catalysts are, for example:

ion-exchange resins having functional groups derived from tertiary amines, quaternary ammonium groups, where hydroxide, chloride or hydrogensulphate may be mentioned by way of example as counterions, sulphonic acid groups or carboxyl groups, where hydrogen, alkali metals or alkaline earth metals may be mentioned by way of example as counterions for both. These functional groups can be bound to the polymer either directly or via inert chains (U.S. Pat. No. 4,062,884 A, U.S. Pat. No. 4,691,041 A, EP 298 167 A). Mention may also be made of alkali metal silicates or alkaline earth metal silicates impregnated onto silicon dioxide supports, and also ammonium-exchanged zeolites.

The production process can be carried out continuously or batchwise. Preference is given to a continuous mode of operation.

The cyclic alkylene carbonate compound(s) and the alkyl alcohol(s) are preferably used in a molar ratio of from 1:0.1 to 1:40, particularly preferably from 1:1.0 to 1:30, very particularly preferably from 1:2.0 to 1:20, in the process. Here, the molar ratio indicated does not take into account the circulation of cyclic alkylene carbonate compound or alcohol to the transesterification column via one or more overhead condenser(s) (cf. under (b)) or one or more bottom vaporizer(s) which may be present.

The catalyst is preferably introduced into the esterification column in dissolved or suspended form together with the stream containing the cyclic alkylene carbonate via a feed point which is located above the feed points for the alkyl alcohol. As an alternative, the catalyst can also be introduced separately, for example as a solution in the alkyl alcohol, in the alkylene glycol or in a suitable inert solvent. When heterogeneous catalysts are used, these can be used in admixture with the abovementioned packing elements, in suitable form instead of packing elements or as a bed on any column trays installed.

The reaction of alkylene carbonate and alkyl alcohol to form dialkyl carbonate and alkylene glycol takes place virtually completely in a transesterification column. In preferred embodiments of the process for preparing dialkyl carbonate, the liquid stream taken off at the bottom of this transesterification column can, if appropriate after being concentrated, be subjected to further reaction and/or purification in one or more further steps. Individual further steps or all such further steps can preferably be carried out in one or more further columns.

As transesterification column or, if used, a second column or further column(s), it is possible to use the columns known to a person skilled in the art. These are, for example, distillation or rectification columns, preferably reactive distillation columns or reactive rectification columns.

The transesterification column preferably contains at least one enrichment section in the upper part of the column and at least one reaction zone below the enrichment section. Each of the two sections preferably has, independently of the other, from 0 to 30, preferably from 0.1 to 30, theoretical plates.

In preferred embodiments, the transesterification column has at least one stripping section below a reaction zone.

The transesterification column can also preferably be equipped with one or more bottom vaporizer(s). When the transesterification column has a stripping section, preference is given to a bottom vaporizer which vaporizes all or part of the liquid flowing down from the stripping section being additionally used. This completely or partially vaporized liquid stream is entirely or partly recirculated to the transesterification column. In the case of an embodiment without a stripping section, the liquid flowing down from the reaction zone is completely or partly vaporized in an optional bottom vaporizer and entirely or partly recirculated to the transesterification column.

The enrichment section(s) can, in preferred embodiments, be accommodated together with the reaction section(s) and if appropriate at least one stripping section in the transesterification column. Here, the gaseous mixture coming from the reaction zone(s) is conveyed from below into a lower region of the enrichment section or if appropriate the lower enrichment section where at least partial removal of the alkylene carbonate or alkylene glycol takes place.

A mixture containing alkylene glycol, excess or unreacted alkylene carbonate, alkyl alcohol, dialkyl carbonate, transesterification catalysts and high-boiling compounds formed in the reaction or present in the starting materials is obtained below the reaction zone and any stripping section present. When a stripping section is used, the content of low-boiling compounds such as dialkyl carbonate and alcohol is reduced, with further dialkyl carbonate and alkylene glycol possibly being formed in the presence of the transesterification catalyst. The energy required for this is preferably supplied by means of one or more vaporizers.

In all sections of the transesterification column, i.e. both in the enrichment section and any stripping section and also in the reaction zone, random packing elements or ordered packings can be used. The packing elements or ordered packings to be used are those customary for distillations, as are described, for example, in Ullmann's Encyclopädie der Technischen Chemie, 4th edition, vol. 2, p. 528 ff. Examples of packing elements are Raschig or PaII or Novalox rings, Berl, Intalex or Torus saddles, Interpack bodies and examples of ordered packings are metal sheets and woven mesh packings, (e.g. BX packings, Montz Pak, Mellapak, Melladur, Kerapak and CY packing) made of various materials such as glass, stoneware, porcelain, stainless steel, plastic. Preference is given to packing elements and ordered packings which have a large surface area, good wetting and a sufficient residence time for the liquid phase. These are, for example, PaII and Novalox rings, Berl saddles, BX packings, Montz Pak, Mellapak, Melladur, Kerapak and CY packings.

As an alternative, it is also possible to use column trays such as sieve trays, bubble cap trays, valve trays, tunnel trays. In the reaction zone(s) of the transesterification column, column trays having high residence times with good mass transfer, for example bubble cap trays, valve trays or tunnel trays having high overflow weirs, are particularly preferred. The number of theoretical plates in the reaction zone is preferably from 3 to 50, particularly preferably from 10 to 50 and very particularly preferably from 10 to 40. The liquid holdup is preferably from 1 to 80%, particularly preferably from 5 to 70% and very particularly preferably from 7 to 60%, of the internal volume of the reaction zone in the column. The more precise design of the reaction zone(s), any stripping section used and the enrichment section(s) can be carried out by a person skilled in the art.

The temperature of the reaction zone(s) is preferably in the range from 20 to 200° C., particularly preferably from 40 to 180° C., very particularly preferably from 50 to 160° C. It is advantageous to carry out the transesterification not only at atmospheric pressure but also at elevated or reduced pressure. The pressure in the reaction zone is therefore preferably in the range from 0.2 to 20 bar, particularly preferably from 0.3 to 10 bar, very particularly preferably from 0.4 to 5 bar. The pressures indicated above and in the following are, unless indicated otherwise, absolute pressures.

The gaseous mixture containing dialkyl carbonate and alkyl alcohol which is taken off at the top of the transesterification column in the process for preparing the dialkyl carbonate is preferably, after condensation at the top of the transesterification column, entirely or partly fed to at least one further process step containing at least one distillation column for separation of dialkyl carbonate and alkyl alcohol.

The separation of the dialkyl carbonate and the alkyl alcohol is preferably carried out by distillation in one or more distillation columns or in a combination of distillation and membrane separation, hereinafter referred to as a hybrid process (see, for example, U.S. Pat. No. 4,162,200 A, EP 581 115 B1, EP 592 883 B1 and WO 2007/096343A1).

If alkyl alcohol and dialkyl carbonate form an azeotrope (e.g. methanol and dimethyl carbonate), it is also possible to use a two-stage process such as a dual pressure process, an extractive distillation, a heteroazeotropic distillation using a low-boiling entrainer or a hybrid process. Particular preference is given to employing the dual pressure process or a hybrid process.

The separation of the dialkyl carbonate and the alkyl alcohol is very particularly preferably carried out in a single distillation column, even when the dialkyl carbonate and the alkyl alcohol form an azeotrope. This distillation column is operated at a pressure which is higher than the pressure of the transesterification column(s). The operating pressure of the distillation column is in the range from 1 to 50 bar, preferably from 2 to 20 bar. Virtually pure dialkyl carbonate is taken off at the bottom of the distillation column and a mixture of dialkyl carbonate and alkyl alcohol is taken off at the top. This mixture is entirely or partly fed to the transesterification column(s). If the process for preparing dialkyl carbonate is coupled with a process for preparing diaryl carbonate formed by transesterification of a dialkyl carbonate with an aromatic hydroxy compound, part of the mixture of dialkyl carbonate and alkyl alcohol taken off at the top of the distillation column can be passed to an appropriate work-up step for alkyl alcohol and dialkyl carbonate in the process step for preparing diaryl carbonate.

In a particularly preferred embodiment when the dialkyl carbonate and the alkyl alcohol form an azeotrope, this work-up step is a dual pressure process. Such processes are known in principle to a person skilled in the art (cf., for example, Ullmann's Encyclopedia of Industrial Chemistry, Vol. 7, 2007, chapter 6.4. and 6.5.; Chemie Ingenieur Technik (67) 11/95).

If alkyl alcohol and dialkyl carbonate form an azeotrope, the distillate from a first distillation column of the process step for separating dialkyl carbonate and alkyl alcohol preferably has a virtually azeotropic composition. In this case, this distillate is preferably fed, in a dual pressure process, to at least one further distillation column which operates at a pressure below that of the first distillation column. The different operating pressure shifts the position of the azeotrope to lower proportions of alkyl alcohol. The bottom product obtained from this second or further distillation column(s) is alkyl alcohol having a purity of from 90 to 100% by weight, based on the total weight of the bottom product isolated, and a virtually azeotropic mixture is obtained as distillate. The second or further distillation column(s) operated at a lower operating pressure is/are, in very particularly preferred embodiments, preferably operated using the heat of condensation of the overhead condenser(s) of the first distillation column.

In the dual pressure process, use is made of the pressure dependence of the azeotropic composition of a two-component mixture. In the case of a mixture of alkyl alcohol and dialkyl carbonate, for example methanol and dimethyl carbonate, the azeotropic composition shifts to higher alkyl alcohol contents with increasing pressure. If a mixture of these two components is fed to a column (dialkyl carbonate column) and the alkyl alcohol content of the mixture is below the azeotropic composition corresponding to the operating pressure of this column, a mixture having a virtually azeotropic composition is obtained as distillate and virtually pure dialkyl carbonate is obtained as bottom product. The azeotropic mixture obtained in this way is fed to a further distillation column (alkyl alcohol column). This operates at a pressure lower than that in the dialkyl carbonate column. As a result, the position of the azeotrope shifts to lower alkyl alcohol contents. This makes it possible for the azotropic mixture obtained in the dialkyl carbonate column to be separated into a distillate having a virtually azeotropic composition and virtually pure alkyl alcohol. The distillate from the alkyl alcohol column is returned to the dialkyl carbonate column at a suitable place.

The operating pressure of the alkyl alcohol column is preferably selected so that the column can be operated using the heat produced by the dialkyl carbonate column. The operating pressure is in the range from 0.1 to 1 bar, preferably from 0.3 to 1 bar. The operating pressure of the dialkyl carbonate column is in the range from 1 to 50 bar, preferably from 2 to 20 bar.

An example of a flow diagram for the separation of dialkyl carbonate and alkyl alcohol by the dual pressure process is shown in FIG. 1.

A further preferred process for the separation of azeotropes of alkyl alcohol and dialkyl carbonate is the hybrid process. In the hybrid process, the separation of a two-component mixture is carried out by means of a combination of distillation and membrane processes. Here, use is made of the fact that the components can be separated at least partly from one another on the basis of their polar properties and their differing molecular weight by means of membranes. In the case of a mixture of alkyl alcohol and dialkyl carbonate, for example methanol and dimethyl carbonate, pervaporation or vapour permeation using suitable membranes gives an alkyl alcohol-rich mixture as permeate and a mixture depleted in alkyl alcohol as retentate. If a mixture of these two components is fed to a column (dialkyl carbonate column) and the alkyl alcohol content of the mixture is below the azeotropic composition corresponding to the operating pressure of this column, a mixture having a significantly increased alkyl alcohol content compared to the feed is obtained as distillate and virtually pure dialkyl carbonate is obtained as bottom product.

In the case of a hybrid process made up of distillation and vapour permeation, the distillate from the column is taken off in vapour form. The gaseous mixture obtained in this way is, if appropriate after superheating, fed to vapour permeation. This is operated with a pressure corresponding to virtually the operating pressure of the column being set on the retentate side and a lower pressure being set on the permeate side. The operating pressure of the column is in the range from 1 to 50 bar, preferably from 1 to 20 bar and particularly preferably from 2 to 10 bar. The pressure on the permeate side is in the range from 0.05 to 2 bar. An alkyl alcohol-rich fraction having an alkyl alcohol content of at least 70%, preferably at least 90%, based on the total weight of the fraction, is obtained on the permeate side. The retentate, which has a reduced alkyl alcohol content compared to the distillate from the column, is condensed if appropriate and recirculated to the distillation column.

In the case of a hybrid process made up of distillation and pervaporation, the distillate from the column is taken off in liquid form. The mixture obtained in this way is, if appropriate after superheating, fed to a pervaporation. This is operated with an identical or increased pressure compared to the column being set on the retentate side and a lower pressure being set on the permeate side. The operating pressure of the column is in the range from 1 to 50 bar, preferably from 1 to 20 bar and particularly preferably from 2 to 10 bar. The pressure on the permeate side is in the range from 0.05 to 2 bar. An alkyl alcohol-rich gaseous fraction having an alkyl alcohol content of at least 70% by weight, preferably at least 90% by weight, based on the total weight of the fraction, is obtained on the permeate side. The liquid retentate, which has a reduced alkyl alcohol content compared to the distillate from the column, is recirculated to the distillation column. Vaporization of the permeate requires heat which may not be available to a sufficient extent in the feed stream to the pervaporation. A membrane separation by means of pervaporation can therefore be heated, if appropriate, by means of additional heat exchangers which are integrated or if appropriate installed between a plurality of pervaporation steps arranged in series.

In the case of a hybrid process, the separation of dialkyl carbonate and alkyl alcohol is particularly preferably carried out by means of a combination of distillation and vapour permeation.

The heat required for the separation of alkyl alcohol and dialkyl carbonate is supplied at a temperature in the range from 100° C. to 300° C., preferably from 100° C. to 230° C. and particularly preferably from 120° C. to 200° C. To allow efficient heat integration with condensers of the diaryl carbonate stage, condensers in which vapours are condensed at a temperature increased by from 1° C. to 100° C., preferably by from 2° C. to 50° C. and particularly preferably by from 5° C. to 40° C., are selected in the diaryl carbonate stage.

In a particularly preferred embodiment, heat energy is obtained from the process steps for preparing diaryl carbonate:
i. intermediate condenser of the first reaction column of the process for preparing diaryl carbonate
ii. overhead condenser of the second reaction column of the process for preparing diaryl carbonate
iii. overhead condenser of the side stream column or condenser in the side stream of the first distillation column for purifying the diaryl carbonate in the process for preparing diaryl carbonate
iv. condenser for the side stream from the second intermediate boiler column of the process for preparing diaryl carbonate.

The heat of condensation from the diaryl carbonate stage which is preferably obtained using the condenser(s) indicated above under i.-iv. can be used, for example, in its entirety or in part for heating, by means of a heat exchanger, one or more column sections of the distillation column for purifying the dialkyl carbonate. In preferred embodiments, the heat of condensation from the condenser(s) of the diaryl carbonate stage indicated above under i.-iv. is used in its entirety or in part for heating, by means of intermediate heaters, the internal liquid stream in the distillation column(s) of the process step for separating dialkyl carbonate and alkyl alcohol.

The intermediate heater can be integrated into the distillation column or be configured as a separate intermediate heater outside the column. The internal or external intermediate heater can have one or more stages (i.e. one or more heat exchangers). In addition, various constructions are possible for the intermediate heater, e.g. integrated heating matrices or heating coils in the case of an internal heater and, for example, plate heat exchangers or shell-and-tube heat exchangers in the case of an external heater. Such constructions are known to those skilled in the art.

In the preferred internal embodiment, the intermediate heater of the distillation column for purifying the dialkyl carbonate preferably has a length of from 100 to 10 000 mm and the ratio of the diameter of the intermediate heater to the column diameter is preferably from 0.1 to 1. Furthermore, the intermediate heater preferably has a heat transfer area of from 1 to 5000 m².

The distillation column(s) for purifying the dialkyl carbonate preferably has/have an enrichment section having preferably from 5 to 40 theoretical plates for concentrating the alkyl alcohol and a stripping section having preferably from 5 to 40 theoretical plates for concentrating the dialkyl carbonate.

The process for preparing dialkyl carbonate is preferably carried out continuously.

The use of the heat of condensation from the condenser(s) of the diaryl carbonate stage indicated under i.-iv. enables the separation of the alkyl alcohol from excess dialkyl carbonate to be carried out with a significantly reduced energy consumption. The cooling power in the diaryl carbonate stage can be reduced to the same degree. A significant advantage of the process described herein compared to the processes of the prior art is therefore the significant reduction in the energy consumption in the preparation of dialkyl carbonates, diaryl carbonates or alkyl aryl carbonates. At the same time, the process can be carried out using simple apparatuses since no complicated reactor arrangement with a plurality of separate reaction zones connected in series is necessary because of the use of column arrangements.

Diaryl carbonates prepared using the processes described herein are preferably those of the general formula (VI)

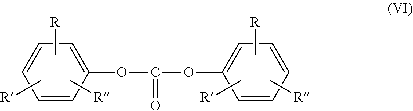

where R, R′ and R″ are each, independently of one another, H, linear or branched, optionally substituted C₁-C₃₄-alkyl, preferably C₁-C₆-alkyl, particularly preferably C₁-C₁-C₃₄-alkoxy, preferably C₁-C₆-alkoxy, particularly preferably C₁-C₄-alkoxy, C₅-C₃₄-cycloalkyl, C₇-C₃₄-alkylaryl, C₆-C₃₄-aryl or a halogen radical, preferably a chlorine radical, and R, R′ and R″ on the two sides of the formula (VI) can be identical or different. R can also be —COO—R″', where R′″ is H, optionally branched C₁-C₃₄-alkyl, preferably C₁-C₆-alkyl, particularly preferably C₁-C₄-alkyl, C₁-C₃₄-alkoxy, preferably C₁-C₆-alkoxy, particularly preferably C₁-C₄-alkoxy, C₅-C₃₄-cycloalkyl, C₇-C₃₄-alkylaryl or C₆-C₃₄-aryl. Preference is given to R, R′ and R″ on the two sides of the formula (VI) being identical. R, R′ and R″ are very particularly preferably each H.

Diaryl carbonates of the general formula (VI) are, for example: diphenyl carbonate, methylphenyl phenyl carbonates and di(methylphenyl)carbonates, also as a mixture, where the methyl group on the phenyl rings can be in any position, and also dimethylphenyl phenyl carbonates and di(dimethylphenyl)carbonates, also as a mixture, where the methyl groups on the phenyl rings can be in any position, chlorophenyl phenyl carbonates and di(chlorophenyl)carbonates, where the methyl group on the phenyl rings can be in any position, 4-ethylphenyl phenyl carbonate, di(4-ethylphenyl)carbonate, 4-n-propylphenyl phenyl carbonate, di(4-n-propylphenyl)carbonate, 4-isopropylphenyl phenyl carbonate, di(4-isopropylphenyl)carbonate, 4-n-butylphenyl phenyl carbonate, di(4-n-butylphenyl)carbonate, 4-isobutylphenyl phenyl carbonate, di(4-isobutylphenyl)carbonate, 4-tert-butylphenyl phenyl carbonate, di(4-tert-butylphenyl)carbonate, 4-n-pentylphenyl phenyl carbonate, di(4-n-pentylphenyl)carbonate, 4-n-hexylphenyl phenyl carbonate, di(4-n-hexylphenyl)carbonate, 4-isooctylphenyl phenyl carbonate, di(4-isooctylphenyl)carbonate, 4-n-nonylphenyl phenyl carbonate, di(4-n-nonylphenyl)carbonate, 4-cyclohexylphenyl phenyl carbonate, di(4-cyclohexylphenyl)carbonate, 4-(1-methyl-1-phenylethyl)phenyl phenyl carbonate, di[4-(1-methyl-1-phenylethyl)phenyl]carbonate, biphenyl-4-yl phenyl carbonate, di(biphenyl-4-yl)carbonate, 1-naphthyl phenyl carbonate, 2-naphthyl phenyl carbonate, di(1-naphthyl)carbonate, di(2-naphthyl)carbonate, 4-(1-naphthyl)phenyl phenyl carbonate, 4-(2-naphthyl)phenyl phenyl carbonate, di[4-(1-naphthyl)phenyl]carbonate, di[4-(2-naphthyl)phenyl]carbonate, 4-phenoxyphenyl phenyl carbonate, di(4-phenoxyphenyl)carbonate, 3-pentadecylphenyl phenyl carbonate, di(3-pentadecylphenyl)carbonate, 4-tritylphenyl phenyl carbonate, di(4-tritylphenyl)carbonate, (methyl salicylate)phenyl carbonate, di(methyl salicylate)carbonate, (ethyl salicylate)phenyl carbonate, di(ethyl salicylate)carbonate, (n-propylsalicylate)phenyl carbonate, di(n-propyl salicylate)carbonate, (isopropyl salicylate)phenyl carbonate, di(isopropyl salicylate)carbonate, (n-butyl salicylate)phenyl carbonate, di(n-butyl salicylate)carbonate, (isobutyl salicylate)phenyl carbonate, di(isobutyl salicylate)carbonate, (tert-butyl salicylate)phenyl carbonate, di(tert-butyl salicylate)carbonate, di(phenyl salicylate)carbonate and di(benzyl salicylate)carbonate.

Preferred diaryl carbonates are: diphenyl carbonate, 4-tert-butylphenyl phenyl carbonate, di(4-tert-butylphenyl)carbonate, biphenyl-4-yl phenyl carbonate, di(biphenyl-4-yl)carbonate, 4-(1-methyl-1-phenylethyl)phenyl phenyl carbonate and di[4-(1-methyl-1-phenylethyl)phenyl]carbonate.

Particular preference is given to diphenyl carbonate.

Aromatic hydroxyl compounds which are suitable for the purposes of the processes described herein are preferably those of the general formula (VII)

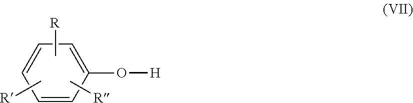

where R, R′ and R″ can each have, independently of one another, the meanings given for the general formula (VI).

Such aromatic hydroxy compounds are, for example: phenol, o-, m- or p-cresol, also as a mixture of the cresols, dimethylphenol, also as a mixture, where the methyl groups on the phenyl ring can be in any positions, e.g. 2,4-, 2,6-, or 3,4-dimethylphenol, o-, m- or p-chlorophenol, o-, m- or p-ethylphenol, o-, m- or p-n-propylphenol, 4-isopropylphenol, 4-n-butylphenol, 4-isobutylphenol, 4-tert-butylphenol, 4-n-pentylphenol, 4-n-hexylphenol, 4-isooctylphenol, 4-n-nonylphenol, o-, m- or p-methoxyphenol, 4-cyclohexylphenol, 4-(1-methyl-1-phenylethyl)phenol, biphenyl-4-ol, 1-naphthol, 2-1-naphthol, 4-(1-naphthyl)phenol, 4-(2-naphthyl)phenol, 4-phenoxyphenol, 3-pentadecylphenol, 4-tritylphenol, methyl salicylate, ethyl salicylate, n-propyl salicylate, isopropyl salicylate, n-butyl salicylate, isobutyl salicylate, tert-butyl salicylate, phenyl salicylate and benzyl salicylate.

Preferred aromatic hydroxy compounds are phenol, 4-tert-butylphenol, biphenyl-4-ol and 4-(1-methyl-1-phenylethyl)phenol.

Particular preference is given to phenol.

Alkyl aryl carbonates prepared by the processes described herein are preferably those of the general formula (VIII)

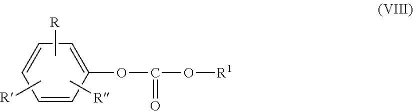

where R, R′ and R″ can have the meanings given for the general formula (VI) and R¹ can have the meanings given for the general formula (I).

Preferred alkyl aryl carbonates are methyl phenyl carbonate, ethyl phenyl carbonate, propyl phenyl carbonate, butyl phenyl carbonate and hexyl phenyl carbonate, methyl o-cresyl carbonate, methyl p-cresyl carbonate, ethyl o-cresyl carbonate, ethyl p-cresyl carbonate, methyl or ethyl p-chlorophenyl carbonate. Particularly preferred alkyl aryl carbonates are methyl phenyl carbonate and ethyl phenyl carbonate. Very particular preference is given to methyl phenyl carbonate.

Both the dialkyl carbonates suitable for the process and the aromatic hydroxy compounds are known to those skilled in the art and are either commercially available or can be prepared by methods which are likewise known to those skilled in the art.

For the purposes of this description, C₁-C₄-alkyl is, for example, methyl, ethyl, n-propyl, isopropyl, n-butyl, sec-butyl, tert-butyl, C₁-C₆-alkyl can also be, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl, C₁-C₃₄-alkyl can also be, for example, n-heptyl and n-octyl, pinacyl, adamantyl, the isomeric menthyls, n-nonyl, n-decyl, n-dodecyl, n-tridecyl, n-tetradecyl, n-hexadecyl or n-octadecyl. The same applies to the corresponding alkyl radical in, for example, aralkyl or alkylaryl radicals. Alkylene radicals in the corresponding hydroxyalkyl or aralkyl or alkylaryl radicals are, for example, the alkylene radicals corresponding to the above alkyl radicals.

Aryl is a carbocyclic aromatic radical having from 6 to 34 skeletal carbon atoms. The same applies to the aromatic part of an arylalkyl radical, also referred to as an aralkyl radical, and also to aryl constituents of more complex groups, e.g. arylcarbonyl radicals.

Arylalkyl or aralkyl is in each case independently a straight-chain, cyclic, branched or unbranched alkyl radical according to the above definition which may be monosubstituted, polysubstituted or persubstituted by aryl radicals according to the above definition.

The listings above are by way of example and not to be construed as a limitation.

It has been found that, in a particularly preferred embodiment a process for preparing at least one diaryl carbonate from at least one dialkyl carbonate and at least one aromatic hydroxy compound, where
(a) the dialkyl carbonate(s) is/are reacted in the presence of at least one transesterification catalyst with the aromatic hydroxy compound(s) in a first reaction column (diaryl carbonate preparation) containing at least one enrichment section in the upper part of the column and at least one reaction zone which is located below the enrichment section and has at least two regions,
(b) the bottom product from the first reaction column (diaryl carbonate preparation) is fed to at least one further reaction column containing at least one enrichment section in the upper part of the column and at least one reaction zone below the enrichment section and is reacted further in the reaction zone,
(c) the dialkyl carbonate which is not reacted in the reaction columns of steps (a) and/or (b) or is formed during the reaction is entirely or partly separated in at least one further process step containing at least one distillation column from alkyl alcohol formed during the reaction,
(d) the vapour containing aromatic hydroxy compound(s) which is taken off at the top of at least one reaction column in (b) is, if appropriate after condensation in at least one condenser, entirely or partly fed to at least one further process step containing at least one distillation column where compounds whose boiling point is between that of the dialkyl carbonate and that of the alkyl aryl carbonate formed during the preparation of the diaryl carbonate are separated off,
(e) the bottom product containing diaryl carbonate which is obtained in the further reaction column(s) in step (b) is fed to at least one further process step for purification in at least one distillation column containing at least one enrichment section in the upper part of the column and at least one stripping section in the lower part of the column,
(f) a catalyst-containing stream is obtained as bottom product from at least one diaryl carbonate distillation column of process step (e) and is entirely or partly recirculated, if appropriate after further purification, to the process, preferably to process step (a),
(g) a stream containing aromatic hydroxy compound(s) and alkyl aryl carbonate is obtained from at least one diaryl carbonate distillation column in process step (e) and is entirely or partly recirculated to the process, preferably to process step (a) or (b), and
(h) compounds having a boiling point above the boiling point of the diaryl

carbonate and compounds whose boiling point lies between that of the dialkyl carbonate and that of the alkyl aryl carbonate formed during the preparation of the diaryl carbonate from at least one diaryl carbonate distillation column in process step (e) are, together or separately from one another, entirely or partly discharged from the process,

and at least one of the reaction column(s) (diaryl carbonate preparation) selected from among the first or the further reaction column(s) is equipped with one or more condensers and the heat of condensation obtained by condensation in these condensers is, directly or indirectly, entirely or partly returned to the process for preparing diaryl carbonate, allows both a work-up of product and waste streams and also efficient energy integration.

The discharge described under (h) can preferably be effected as a liquid side stream from the enrichment section of at least one diaryl carbonate distillation column and/or a substream of the distillate from this column.

In the process for preparing diaryl carbonate, the aromatic hydroxy compound(s) and the dialkyl carbonate(s) are preferably used in a molar ratio of from 1:0.1 to 1:10, particularly preferably from 1:0.2 to 1:5, very particularly preferably from 1:0.5 to 1:3, in the first reaction column (diaryl carbonate preparation). The molar ratio indicated does not take into account the recirculation of aromatic hydroxy compound or dialkyl carbonate to the reaction column via one or more overhead condenser(s) (cf. under (b)) or one or more bottom vaporizers which may be present.

The process for preparing diaryl carbonates is carried out in at least two reaction columns.

Columns known to those skilled in the art are possible as first and second reaction column or, if used, third or further column(s). These are, for example, distillation or rectification columns, preferably reactive distillation columns or reactive rectification columns.

The first reaction column (diaryl carbonate preparation) contains at least one enrichment section in the upper part of the column and at least one reaction zone which is located below the enrichment section and has at least two regions. Preference is given to each of the two regions independently having from 0 to 20 theoretical plates, preferably from 0.1 to 20 theoretical plates. In preferred embodiments, at least one enrichment section of the first reaction column is equipped with at least one intermediate condenser. The intermediate condenser is preferably installed between the two regions of the enrichment section. In this case, the enrichment section is divided into an upper enrichment section and a lower enrichment section.

The first reaction column (diaryl carbonate preparation) is preferably operated in countercurrent, with preference being given to the aromatic hydroxy compound being introduced in liquid form from the top to the bottom in at least one reaction zone of this column and the dialkyl carbonate being conveyed in gaseous form in countercurrent to this liquid stream. The first reaction column is in this case preferably operated with one or more streams containing the aromatic hydroxy compound and if appropriate dissolved transesterification catalyst being introduced in liquid form or with only a small proportion of gas, with the proportion of gas preferably being less than 20% by weight, into at least one reaction zone, preferably into the upper third of the reaction zone, preferably at the temperature prevailing at this point in the column. In addition, one or more streams containing the dialkyl carbonate are introduced into the reaction zone, preferably the lower third of this reaction zone, with the introduction preferably taking place in gaseous or superheated form. In preferred embodiments, the superheating of the vapour stream can be from 0 to 50° C. Furthermore, the dew point temperature is preferably determined by the pressure prevailing in the reaction zone at the point of introduction of the respective dialkyl carbonate.

After passing through the reaction zone(s), the alkyl alcohol formed during the reaction is taken off at the top of the first reaction column (diaryl carbonate preparation) after passing through the enrichment section(s). The alkyl alcohol formed during the reaction is the alcohol liberated in the transesterification, preferably R¹—OH or R²—OH, where R¹ and R² are as defined for the general formula (I). The stream taken off at the top of the first reaction column generally contains not only the alkyl alcohol formed in the reaction but also excess or unreacted dialkyl carbonate and low-boiling secondary compounds such as carbon dioxide or dialkyl ether. Owing to the enrichment section(s) present, this stream contains only small amounts of higher-boiling components such as the aromatic hydroxy compound. The enrichment section serves to separate off the relatively high-boiling components which are also vaporized in the reaction zone, e.g. the aromatic hydroxy compound or alkyl aryl carbonate, from the low-boiling alkyl alcohols or dialkyl carbonates. This has the advantage that the separation of the alkyl alcohols formed during the reaction from the dialkyl carbonates can be carried out at a low temperature level.

The first reaction column (diaryl carbonate preparation) is, in preferred embodiments, operated under reflux conditions. As used herein, reflux conditions refers to a mode of operation in which the vapour stream at the upper end of the enrichment section is partly or completely condensed (cf. under (b)) and the condensate obtained is partly or completely returned as runback to the upper end of the enrichment section. The reflux ratio is preferably from 0.1 to 20, particularly preferably from 0.1 to 10 and very particularly preferably from 0.1 to 3, with the reflux ratio corresponding, for the purposes of this description, to the weight ratio of condensate returned to the column to vapour taken off at the top of the column without returned condensate.

In preferred embodiments, the first reaction column (diaryl carbonate preparation) has at least one stripping section below a reaction zone.

The first reaction column (diaryl carbonate preparation) can also preferably be equipped with one or more bottom vaporizer(s). When the first reaction column has a stripping section, preference is given to additionally using a bottom vaporizer which completely or partly vaporizes the liquid running down from the stripping section. This totally or partly vaporized liquid stream is entirely or partly recirculated to the first reaction zone. In the case of an embodiment without a stripping section, the liquid flowing down from the reaction zone is entirely or partly vaporized in a bottom vaporizer which may be used and recirculated entirely or partly to the first reaction column.

In the preferred embodiments in which at least one enrichment section of the first reaction column (diaryl carbonate preparation) is equipped with at least one intermediate condenser, the enrichment section of the first reaction column which is equipped with at least one intermediate condenser is divided into a lower enrichment section and an upper enrichment section (two regions) of which the lower enrichment section is located below the intermediate condenser and the upper enrichment section is located above the intermediate condenser.

The enrichment section(s) having at least one intermediate condenser can, in preferred embodiments, be accommodated together with the reaction section(s) and, if appropriate, at least one stripping section in the reaction column. The gaseous mixture coming from the reaction zone(s) is introduced from below into a lower region of the enrichment section or, if appropriate, into the lower enrichment section where at least partial removal of the aromatic hydroxy compound takes place. The gaseous mixture coming from this lower region or, if appropriate, the lower enrichment section is introduced into an intermediate condenser where it is partly condensed out and the condensate obtained is fed to the upper end of the lower region of the enrichment section or, if appropriate, the lower enrichment section.

In a further preferred embodiment of the process, the intermediate condenser is not integrated into the first reaction column (diaryl carbonate preparation) but configured as a separate intermediate condenser outside the first reaction column.

In a further preferred embodiment of the process, intermediate condenser and the upper region of the enrichment section are not integrated into the reaction column (diaryl carbonate preparation) but instead accommodated separately outside the first reaction column.

A mixture containing alkyl aryl carbonate, excess or unreacted phenol, diaryl carbonate, transesterification catalysts, dialkyl carbonate, alkyl alcohol and high-boiling compounds formed in the reaction or originally present in the starting materials is obtained below the reaction zone and any stripping section present. When a stripping section is used, the content of low-boiling compounds such as dialkyl carbonate and alkyl alcohol is reduced, and further alkyl aryl carbonate and/or diaryl carbonate can possibly be formed in the presence of the transesterification catalyst. The necessary energy is preferably supplied by one or more vaporizers.

In all sections of the first reaction column (diaryl carbonate preparation), i.e. both in the enrichment section and any stripping section and also in the reaction zone, random packing elements or ordered packings can be used. The packing elements or ordered packings to be used are those customary for distillations, as are described, for example, in Ullmann's Encyclopadie der Technischen Chemie, 4^{th} edition, Vol. 2, p. 528 ff. Examples of packing elements are Raschig or PaII and Novalox rings, Berl, Intalex or Torus saddles, Interpack bodies, and examples of ordered packings are sheet metal and woven mesh packings (e.g. BX packings, Montz Pak, Mellapak, Melladur, Kerapak and CY packing) made of various materials such as glass, stoneware, porcelain, stainless steel, plastic. Preference is given to packing elements and ordered packings which have a large surface area, good wetting and sufficient residence time for the liquid phase. These are, for example, PaII and Novalox rings, Berl saddles, BX packings, Montz Pak, Mellapak, Melladur, Kerapak and CY packings.

As an alternative, column trays such as sieve trays, bubble cap trays, valve trays and tunnel trays are also suitable. Column trays having high residence times with good mass transfer, for example bubble cap trays, valve or tunnel trays with high overflow weirs, are particularly preferred in the reaction zone(s) of the reaction column (diaryl carbonate preparation). The number of theoretical plates in the reaction zone is preferably from 3 to 50, particularly preferably from 10 to 50 and very particularly preferably from 10 to 40. The liquid holdup is preferably from 1 to 80%, particularly preferably from 5 to 70% and very particularly preferably from 7 to 60%, of the internal volume of the column in the reaction zone. The more precise design of the reaction zone(s), any stripping section to be used and the enrichment section(s) can be carried out by a person skilled in the art.

The temperature of the reaction zone(s) is preferably in the range from 100 to 300° C., particularly preferably from 120 to 250° C., very particularly preferably from 150 to 240° C. In preferred embodiments, an optimal reaction temperature is set in the reaction zone, firstly by choice of the operating conditions and secondly by additional introduction of heat in the region of one or more reaction trays. Heat can be introduced to the reaction trays either by means of heat exchangers or by means of reaction trays having facilities for introduction of heat. It is advantageous to carry out the transesterification not only at atmospheric pressure but also at superatmospheric or subatmospheric pressure. The pressure in the reaction zone is therefore preferably in the range from 0.5 to 20 bar (absolute), particularly preferably from 0.8 to 15 bar (absolute), very particularly preferably from 0.9 to 10 bar (absolute).

Transesterification catalysts known from the literature can be used for the reaction steps occurring in the first reaction column (diaryl carbonate preparation). These are transesterification catalysts known from the literature for the dialkyl carbonate-phenol transesterification, e.g. AlX₃, TiX₃, UX₄, TiX₄, VOX₃, VX₅, ZnX₂, FeX₃, PbX₂ and SnX₄, where X is a halogen, acetoxy, alkoxy or aryloxy radical (DE-A 2 58 412). Particularly preferred catalysts are metal compounds such as AlX₃, TiX₄, PbX₂ and SnX₄, for example titanium tetrachloride, titanium tetramethoxide titanium tetraphenoxide, titanium tetraethoxide, titanium tetraisopropoxide, titanium tetradodecoxide, tin tetraisooctoxide and aluminium triisopropoxide. Very particular preference is given to metal compounds TiX₄. The metal compounds mentioned are preferably used in amounts of from 0.001 to 5% by weight, preferably from 0.005 to 5% by weight and particularly preferably from 0.01 to 5% by weight, based on the weight of the reaction mixture to be reacted.

For the purposes of this description, halogen is fluorine, chlorine or bromine, preferably fluorine or chlorine, particularly preferably chlorine.

Further catalysts which can be used are organotin compounds of the general formula (R¹¹)^{4-x}—Sn(Y)ₓ, where Y is a radical OCOR¹², OH or OR, where R¹² is C₁-C₁₂-alkyl, C₆-C₁₂-aryl or C₇-C₁₃-alkylaryl, the radicals R¹¹ each have, independently of R¹², one of the meanings of R¹² and x is an integer from 1 to 3, dialkyltin compounds having from 1 to 12 carbon atoms in the alkyl radical or bis(trialkyltin) compounds, for example trimethyltin acetate, triethyltin benzoate, tributyltin acetate, triphenyltin acetate, dibutyltin diacetate, dibutyltin dilaurate, dioctyltin dilaurate, dibutyltin adipate, dibutyldimethoxytin, dimethyltin glycolate, dibutyldiethoxytin, triethyltin hydroxide, hexaethylstannoxane, hexabutylstannoxane, dibutyltin oxide, dioctyltin oxide, butyltin triisooctoxide, octyltin triisooctoxide, butylstannonoic acid and octylstannonoic acid in amounts of from 0.001 to 20% by weight (cf. EP 879 A, EP 880 A, EP 39 452 A, DE-A 34 45 555, JP 79/63023), polymeric tin compounds of the formula —[—RR¹¹Sn—O—]—, where R and R¹¹ each have, independently of one another, one of the meanings given above for R¹², for example poly[oxy(dibutylstannylene)]poly[oxy(dioctylstannylene)], poly[oxy(butylphenyl-stannylene)] and poly[oxy(diphenylstannylene)] (DE-A 34 45 552), polymeric hydroxystannoxanes of the formula —[—RSn(OH)—O—]—, for example poly(ethylhydroxystannoxane), poly(butylhydroxystannoxane), poly(octylhydroxystannoxane), poly(undecylhydroxystannoxane) and poly(dodecylhydroxystannoxane) in amounts of from 0.001 to 20% by weight, preferably from 0.005 to 5% by weight, based on dialkyl carbonate (DE-A 40 06 520). Further tin compounds which can be used are Sn(II) oxides of the general formula

X—R₂Sn—O—R₂Sn—Y,

where X and Y are each, independently of one another, OH, SCN, OR¹³, OCOR¹³ or halogen and R is alkyl, aryl, where R¹³ has the meaning given above for R¹² (EP 0 338 760).

Further catalysts which can be used are lead compounds, if appropriate together with triorganophosphanes, a chelating compound or an alkali metal halide, for example Pb(OH)₂-2PbCO₃, Pb(OCO—CH₃)₂, Pb(OCO—CH₃)₂.2LiCl, Pb(OCO—CH₃)₂.2PPh₃ in amounts of from 0.001 to 1 mol, preferably from 0.005 to 0.25 mol, per mole of dialkyl carbonate (JP 57/176932, JP 01/093580) and also other lead(II) and lead(IV) compounds such as PbO, PbO₂, minimum, plumbites and plumbates (JP 01/093560), iron(III) acetate (JP 61/1 72 852), also copper salts and/or metal complexes, for example of alkali metals, zinc, titanium and iron (JP 89/005588).

Furthermore, heterogeneous catalyst systems can be used in the processes. Examples of such systems are mixed oxides of silicon and titanium which can be obtained by joint hydrolysis of silicon and titanium halides (JP 54/125617) or titanium dioxides having a high BET surface area of >20 m²/g (DE-A 40 36 594)).

Preferred catalysts for the process are the abovementioned metal compounds AlX₃, TiX₃, UX₄, TiX₄, VOX₃, VX₅, ZnX₂, FeX₃, PbX₂ and SnX₄. Particular preference is given to AlX₃, TiX₄, PbX₂ and SnX₄, among which mention may be made by way of example of titanium tetrachloride, titanium tetramethoxide, titanium tetraphenoxide, titanium tetraethoxide, titanium tetraisopropoxide, titanium tetradodecoxide, tin tetraisooctoxide and aluminium triisopropoxide. Very particular preference is given to metal compounds TiX₄. In particular, preference is given to titanium tetramethoxide, titanium tetraphenoxide and titanium tetraethoxide.

The catalyst is preferably introduced in dissolved or suspended form together with the stream containing the aromatic hydroxy compound(s) into the first reaction column (diaryl carbonate preparation). As an alternative, the catalyst can also be introduced separately, for example in an alcohol corresponding to the alkyl alcohol or in a suitable inert solvent. When heterogeneous catalysts are used, these can be used in admixture with the packing elements mentioned, in suitable form instead of packing elements or as a bed on any column trays installed.

The energy required for the reaction in the first reaction column (diaryl carbonate preparation) can be generated via internal or external devices such as heat exchangers, vaporizers and/or heatable column trays and/or be introduced either with the liquid stream containing the aromatic hydroxy compound(s) or with the dialkyl carbonate-containing stream which is introduced in gaseous form. Particularly in the region of the reaction zone(s), heat can be introduced in this way. This heat is preferably introduced in the region of the reaction zone(s) entirely or partly by means of vaporizers or heatable column trays. It is particularly advantageous to introduce the energy required for the reaction in the first reaction column at least partly both with the liquid stream containing the aromatic hydroxy compound(s) and with the dialkyl carbonate-containing stream introduced in gaseous form into the first reaction column and additionally by means of internal and/or external heat exchangers.

In the process, the bottom product from the first reaction column is fed to a second reaction column.

The second reaction column (diaryl carbonate preparation) contains at least one enrichment section in the upper part of the column and at least one reaction zone below the enrichment section. The enrichment section preferably has from 1 to 50, particularly preferably from 1 to 25, theoretical plates.

In the second reaction column (diaryl carbonate preparation), the bottom product from the first reaction column (diaryl carbonate preparation), which contains alkyl aryl carbonate and diaryl carbonate already formed, is preferably fed in liquid form or as a vapour/liquid mixture into the reaction zone, particularly preferably the upper part of the reaction zone, very particularly preferably in the upper third of the reaction zone. The second reaction column is preferably operated so that the alkyl aryl carbonate is partly or completely converted into the diaryl carbonate, for example by further transesterification or disproportionation, preferably by disproportionation. In addition to the bottom product from the first reaction column, one or more streams containing alkyl aryl carbonate can be introduced in liquid form or as a vapour/liquid mixture in the region of the reaction zone. Such additional streams containing alkyl aryl carbonate can come, for example, from the further work-up and be recirculated to the process in this way.

At the top of the second reaction column, unreacted aromatic hydroxy compound, dialkyl carbonate, alkyl alcohol, intermediate-boiling secondary compounds such as alkyl aryl ethers and, to a small extent, low-boiling secondary compounds are separated off. For the purposes of this description, intermediate-boiling secondary compounds are those having a boiling point below that of the aromatic hydroxy compound and above that of the dialkyl carbonate. Such intermediate-boiling secondary compounds are, for example, alkyl aryl ethers such as anisole or phenetole. The intermediate-boiling secondary compounds separated off in the second reaction column can arise in the reaction in the first and/or second reaction column or have been introduced into the process via the starting materials.

The enrichment section of the second reaction column (diaryl carbonate preparation) serves to separate off the relatively high-boiling components which are also vaporized in the reaction zone, e.g. alkyl aryl carbonate.

The second reaction column (diaryl carbonate preparation) is operated under reflux conditions in preferred embodiments. As used herein, reflux conditions refer to a mode of operation in which the vapour stream at the upper end of the enrichment section is partly or fully condensed and the condensate obtained is partly or entirely returned as runback to the upper end of the enrichment section. The reflux ratio is preferably from 0.1 to 20, particularly preferably from 0.1 to 10 and very particularly preferably from 0.1 to 3, with the reflux ratio corresponding, for the purposes of this description, to the weight ratio of condensate returned to the column to vapour taken off at the top of the column without returned condensate.

The second reaction column (diaryl carbonate preparation) can have at least one stripping section below a reaction zone. However, in preferred embodiments, the reaction zone of the second reaction column can simultaneously function as stripping section. In this case, the dialkyl carbonate liberated in the disproportionation, alkyl alcohol liberated by transesterification and unreacted aromatic hydroxy compound are separated off and at the same time diaryl carbonate and the alkyl aryl carbonate which reacts essentially by disproportionation are concentrated.

The second reaction column (diaryl carbonate preparation) can also preferably be equipped with one or more bottom vaporizer(s).

In principle, the enrichment section of the second reaction column (diaryl carbonate preparation) can likewise be equipped with one or more intermediate condensers. In this way, the enrichment section is divided into a lower enrichment section and an upper enrichment section (two regions), of which the lower enrichment section is located below the intermediate condenser and the upper enrichment section is located above the intermediate condenser. In a preferred embodiment, the second reaction column has no intermediate condenser.

The second reaction column (diaryl carbonate preparation) is equipped with one or more condensers. Preference is given to one or more condensers being located at the top of the second reaction column (overhead condenser(s)). Particular preference is given to using a cascade of overhead condensers.

During the course of the condensation in the condenser(s) at the top of the second reaction column, the vapours become depleted in relatively high-boiling components such as aromatic hydroxy compound. To be able to utilise the heat of condensation evolved in terms of heat integration particularly efficiently, the condensation is preferably carried out in a plurality of stages, particularly preferably in at least two stages, in preferred embodiments in two or three stages.

In the particularly preferred embodiment of two- or three-stage condensation, the heat of condensation from the first condensation stage or the first and second condensation stages is used directly or indirectly for heating a stream or a column within the process, while the heat of condensation obtained from the second or third condensation stage is removed by means of cooling water or air cooling.

The condensation at the top of the second reaction column can, in further preferred embodiments, also be carried out so that part of the vapours taken off at the top of the second reaction column is not condensed in order to be able to discharge intermediate-boiling secondary compounds selectively.

A mixture containing alkyl aryl carbonate, excess or unreacted aromatic hydroxy compound, diaryl carbonate, transesterification catalyst(s), dialkyl carbonate, alkyl alcohol and intermediate- or high-boiling secondary compounds formed in the reaction or originally present in the starting materials is obtained below the reaction zone and any stripping section present. For the purposes of this description, high-boiling secondary compounds are those having a boiling point above that of the aromatic hydroxy compound.

In all sections of the second reaction column (diaryl carbonate preparation), i.e. both in the enrichment section and any stripping section and also in the reaction zone, it is possible to use random packing elements or ordered packings. The packing elements or ordered packings to be used are those customary for distillations, as are described, for example, in Ullmann's Encyclopadie der Technischen Chemie, 4th edition, Vol. 2, p. 528 ff. Examples of packing elements are Raschig or PaII and Novalox rings, Berl, Intalex or Torus saddles, Interpack bodies and examples of ordered packings are sheet metal and woven mesh packings (e.g. BX packings, Montz Pak, Mellapak, Melladur, Kerapak and CY packing) made of various materials such as glass, stoneware, porcelain, stainless steel, plastic. Preference is given to packing elements and ordered packings which have a large surface area, good wetting and sufficient residence time of the liquid phase. These are, for example, PaII and Novolax rings, Berl saddles, BX packings, Montz Pak, Mellapak, Melladur, Kerapak and CY packings.

As an alternative, column trays such as sieve trays, bubble cap trays, valve trays, tunnel trays are also suitable. In the reaction zone(s) of the second reaction column (diaryl carbonate preparation), beds of random packing elements or structured packings are particularly preferred. The number of theoretical plates in the reaction zone is preferably from 3 to 50, particularly preferably from 10 to 50 and very particularly preferably from 10 to 40.

The more precise design of the reaction zone(s), any stripping section to be used and the enrichment section(s) can be carried out by a person skilled in the art.

The temperature of the reaction zone(s) is preferably in the range from 100 to 300° C., particularly preferably from 120 to 250° C., very particularly preferably from 180 to 250° C.

In particular embodiments, an optimal reaction temperature is set in the reaction zone firstly by choice of the operating conditions and secondly by means of additional introduction of heat in the region of one or more reaction trays. The supply of heat to the reaction trays can be effected either by means of heat exchangers or by means of reaction trays having facilities for introduction of heat. It is advantageous to carry out the transesterification not only at atmospheric pressure but also at superatmospheric or subatmospheric pressure, preferably at subatmospheric pressure. The pressure in the second reaction column (diaryl carbonate preparation) is therefore preferably in the range from 0.05 to 20 bar (absolute), particularly preferably from 0.1 to 10 bar (absolute), very particularly preferably from 0.1 to 2 bar (absolute).

The transesterification catalysts which have been mentioned above for the transesterification in the first reaction column can be used for the reaction steps occurring in the second reaction column (diaryl carbonate preparation). In a preferred embodiment, identical catalysts are used in the first and second reaction columns.

The catalyst is preferably introduced in dissolved or suspended form together with the bottom product from the first reaction column (diaryl carbonate preparation) into the second reaction column (diaryl carbonate preparation). As an alternative, the catalyst can also be introduced separately, for example in an alcohol corresponding to the alkyl alcohol or in a suitable inert solvent. When heterogeneous catalysts are used, these can be used in admixture with the packing elements mentioned, in suitable form in place of packing elements or as a bed on column trays which may be installed.

The energy required for the reaction in the second reaction column can be generated by means of internal or external devices such as heat exchangers, vaporizers and/or heatable column trays and/or be introduced with the liquid stream containing the aromatic hydroxy compound(s). This heat is preferably introduced in the region of the reaction zone(s) either entirely or partly by means of vaporizers.

The second reaction column can be followed by one or more further reaction columns. The conditions and parameter ranges indicated above for the second reaction column apply to such further reaction columns, but the conditions and parameters of further reaction columns do not have to be identical to those in the second reaction column but preferably differ from those in the second reaction column within the abovementioned ranges of conditions and parameters. A reaction column in addition to the second reaction column is preferably operated, for example, at a lower pressure than the second reaction column; reflux ratio and temperature at the bottom can also differ from those in the second reaction column. In a preferred embodiment, the first reaction column in the process is followed by only one further reaction column, i.e. the abovementioned second reaction column. However, further columns for purification and separation of the components of the streams taken off can follow the reaction columns. Such columns for purification and separation of the components are not reaction columns for the purposes of this description.

In the process for preparing diaryl carbonate, streams containing alkyl alcohol formed during the reaction and also unreacted dialkyl carbonate or dialkyl carbonate formed during the reaction are obtained in the transesterification and/or disproportionation in the first reaction column (diaryl carbonate preparation) and/or the further reaction column(s) and are preferably taken off in admixture in one or more streams. This dialkyl carbonate which has not been reacted in the reaction columns or has been formed during the reaction is entirely or partly separated in at least one further process step containing at least one distillation column from alkyl alcohol formed during the reaction. Preference is given to taking off at least one stream containing unreacted dialkyl carbonate or dialkyl carbonate formed during the reaction and alkyl alcohol formed during the reaction at the top of the first reaction column (diaryl carbonate preparation) and feeding it to at least one further process step containing at least one distillation column for the purpose of separation.

The vapour mixture containing dialkyl carbonate and alkyl alcohol formed during the reaction which is taken off at the top of the first reaction column (diaryl carbonate preparation) is preferably, after condensation at the top of the first reaction column, entirely or partly fed to at least one further process step containing at least one distillation column for separation of dialkyl carbonate and alkyl alcohol, hereinafter referred to as separation distillation column(s). The dialkyl carbonate separated off here is particularly preferably recirculated, if appropriate after further purification, to the first reaction column.

The separation of the dialkyl carbonate and the alkyl alcohol is preferably carried out by distillation in one or more separation distillation columns or in a combination of distillation and membrane separation, also referred to as hybrid process.

The separation of the dialkyl carbonate and the alkyl alcohol is carried out in a manner analogous to that described above for the dialkyl carbonate stage. The vapour containing aromatic hydroxy compound(s) which is taken off at the top of at least one reaction column (diaryl carbonate preparation) of (b), if appropriate after condensation in at least one condenser, is entirely or partly fed to at least one further process step containing at least one distillation column where compounds whose boiling point lies between that of the dialkyl carbonate and that of the alkyl aryl carbonate formed during the preparation of the diaryl carbonate, hereinafter also referred to as intermediate-boiling compounds, are separated off. The distillation column(s) used in this process step for separating off compounds whose boiling point lies between that of the dialkyl carbonate and the alkyl aryl carbonate formed during the preparation of the diaryl carbonate are hereinafter referred to as intermediate boiler distillation columns.

In a preferred embodiment of the process for preparing diaryl carbonate, the vapour containing aromatic hydroxy compound(s) which is taken off at the top of at least one reaction column of (b), if appropriate after condensation in at least one condenser, is fed to at least one further process step containing at least two intermediate boiler distillation columns, with the bottom product from the first intermediate boiler distillation column being fed to a second intermediate boiler distillation column.

The aromatic hydroxy compound(s) obtained from the vapour containing aromatic hydroxy compound(s) which is taken off entirely or partly at the top of at least one reaction column of (b), if appropriate after condensation in at least one condenser, in the process step(s) for separating off compounds whose boiling point lies between that of the dialkyl carbonate and the alkyl aryl carbonate formed during the preparation of the diaryl carbonate is (are) preferably returned to the first reaction column. The aromatic hydroxy compound(s) obtained after the separation is (are) preferably taken off from a first and only intermediate boiler distillation column as bottom product or from a second or further intermediate boiler distillation column as side stream or bottom product.

The product taken off at the top of the first intermediate boiler distillation column preferably contains dialkyl carbonate and is entirely or partly fed to the process step (c) containing at least one separation distillation column to separate off the alkyl alcohol.

In a preferred embodiment, alkyl alcohol, dialkyl carbonate and possibly part of the intermediate-boiling secondary components are separated off as overhead product in the first intermediate boiler distillation column(s). In this case, this preferably has an enrichment section having from 5 to 40 theoretical plates for concentrating the alkyl alcohol and the dialkyl carbonate and a stripping section having from 5 to 40 theoretical plates for concentrating the intermediate-boiling secondary compounds. The operating pressure is preferably in the range from 0.05 to 3 bar absolute, particularly preferably from 0.1 to 2 bar absolute and very particularly preferably from 0.5 to 1.5 bar absolute. The reflux ratio is preferably from 0.1 to 10, particularly preferably from 0.5 to 5 and very particularly preferably from 0.5 to 2.

In the case of the abovementioned preferred embodiment of the first intermediate boiler distillation column, the aromatic hydroxy compound is taken off as bottom product or side stream, particularly preferably as side stream, intermediate-boiling secondary compounds having a boiling point above that of the aromatic hydroxy compound are taken off at the bottom and intermediate-boiling secondary compounds having a boiling point below that of the aromatic hydroxy compound are taken off as distillate. In the case of this preferred embodiment, the column preferably has an enrichment section having at least one region and a separating power of from 5 to 40 theoretical plates, a stripping section having preferably at least one, particularly preferably at least 2, region(s) and a separating power of from 5 to 60 theoretical plates. The operating pressure is preferably in the range from 0.05 to 3 bar absolute, particularly preferably from 0.1 to 2 bar absolute and very particularly preferably from 0.5 to 1.5 bar absolute. The reflux ratio is preferably from 1 to 1000, particularly preferably from 10 to 500 and very particularly preferably from 50 to 200.

In the case of a particularly preferred embodiment of the second intermediate boiler distillation column, the aromatic hydroxy compound is taken off as gaseous side stream. The heat obtained in the condensation of the gaseous side stream can be utilized either for generating a heat transfer medium or directly for heating other process steps for preparing diaryl carbonates.

The bottom product containing diaryl carbonate which is obtained in the further reaction column(s) of step (b) is fed to at least one further process step for purification in at least one distillation column, hereinafter also referred to as first diaryl carbonate distillation column, containing at least one enrichment section in the upper part of the column and at least one stripping section in the lower part of the column. A diaryl carbonate-containing side stream is preferably taken off from this first diaryl carbonate distillation column. Furthermore, the bottom product containing diaryl carbonate which is obtained in the further reaction column(s) of step (b) preferably contains compounds having a boiling point between that of the diaryl carbonate and that of the alkyl aryl carbonate formed as intermediate during the preparation of the diaryl carbonate as impurities which are taken off in a further side stream from the diaryl carbonate distillation column and are optionally recirculated to (one of) the further reaction column(s) of step (b).

The bottom product obtained in the further reaction column(s) of step (b), also referred to as crude diaryl carbonate, preferably contains from 10 to 90% by weight, particularly preferably from 20 to 80% by weight and very particularly preferably from 40 to 80% by weight, of diaryl carbonate and from 5 to 90% by weight, particularly preferably from 5 to 60% by weight and very particularly preferably from 5 to 40% by weight, of alkyl aryl carbonate, from 1 to 90% by weight, particularly preferably from 1 to 50% by weight and very particularly preferably from 1 to 30% by weight, of aromatic hydroxy compound, from 0 to 5% by weight, particularly preferably from 0 to 2% by weight and very particularly preferably from 0 to 0.5% by weight of high-boiling secondary components, from 0 to 5% by weight, particularly preferably from 0.0001 to 2% by weight and very particularly preferably from 0.0001 to 1% by weight, of intermediate-boiling secondary components and from 0.01 to 10% by weight, particularly preferably from 0.1 to 5% by weight and very particularly preferably from 1 to 5% by weight, of catalyst, where the sum of all the abovementioned components in the diaryl carbonate to be purified is 100% by weight. The % by weight figures are in each case based on the total weight of the crude diaryl carbonate to be purified.

The process preferably makes it possible to obtain diaryl carbonates having a purity of, i.e. a content of pure diaryl carbonate of, from 99 to 100% by weight, particularly preferably from 99.5 to 100% by weight and very particularly preferably from 99.9 to 100% by weight, based on the total weight of the purified diaryl carbonate.

The diaryl carbonate taken off in the side stream from the first diaryl carbonate distillation column can be taken off in liquid or vapour form. The diaryl carbonate taken off in the side stream from the first diaryl carbonate distillation column is preferably taken off in vapour form. However, in preferred embodiments, taking off the diaryl carbonate in liquid form in the side stream can be preferred, especially as a result of constructional circumstances.

The first diaryl carbonate distillation column has at least two regions, i.e. an enrichment section in the upper part of the column and a stripping section in the lower part of the column. The enrichment section of the first diaryl carbonate distillation column can preferably be divided into a lower enrichment section and an upper enrichment section. Furthermore, the stripping section of the first diaryl carbonate distillation column can preferably be divided into a lower stripping section and an upper stripping section.

In a preferred embodiment of the process for preparing diaryl carbonate, the purification of the bottom product containing diaryl carbonate which is obtained in the further reaction column(s) of step (b) is carried out in at least one diaryl carbonate distillation column which has at least three regions. These at least three regions are at least one enrichment section and at least one stripping section, with the stripping section being divided into a lower stripping section and an upper stripping section. The first diaryl carbonate distillation column having an enrichment section and a stripping section, with the stripping section being divided into a lower stripping section and an upper stripping section, particularly preferably has four regions, with the enrichment section also being divided into a lower enrichment section and an upper enrichment section.

The first diaryl carbonate distillation column preferably has a total separating power of from 3 to 160, particularly preferably from 10 to 90, very particularly preferably from 13 to 50, theoretical plates. The upper enrichment section preferably has a separating power of from 0 to 40, particularly preferably from 1 to 20, very particularly preferably from 1 to 10, theoretical plates, the lower enrichment section preferably has from 1 to 40, particularly preferably from 5 to 20, very particularly preferably from 5 to 15, theoretical plates, the upper stripping section preferably has from 1 to 40, particularly preferably from 2 to 30, very particularly preferably from 5 to 20, theoretical plates and the lower stripping section preferably has from 1 to 40, particularly preferably from 2 to 20, very particularly preferably from 2 to 15, theoretical plates.

Vaporization is preferably carried out in a temperature range from 100 to 300° C., preferably from 150 to 240° C. and particularly preferably from 180 to 230° C., in the bottom of the column. The condensation of the vapours at the top of the column can be effected in one or more stages, preferably one or two stages, in a temperature range of preferably from 40 to 250° C., preferably from 50 to 200° C. and particularly preferably from 60 to 180° C.

The first diaryl carbonate distillation column is preferably operated at a pressure at the top of from 1 to 1000 mbar (absolute), particularly preferably from 1 to 100 mbar (absolute) and very particularly preferably from 5 to 50 mbar (absolute). The reflux ratio is preferably from 0.1 to 10, particularly preferably from 0.5 to 5 and very particularly preferably from 0.5 to 2.

In a further particularly preferred embodiment of the process, the diaryl carbonate taken off in the side stream from the first diaryl carbonate distillation column is purified in at least one, preferably in a second, diaryl carbonate distillation column. In a particularly preferred variant of this preferred embodiment, this second diaryl carbonate distillation column does not have a stripping section.

In this particularly preferred variant, the diaryl carbonate is purified in a first diaryl carbonate distillation column and an additional side stream column, viz. the second diaryl carbonate distillation column. The gaseous side stream from the first diaryl carbonate distillation column is fed to the side stream column, preferably to the lower part thereof. The liquid bottom product from the side stream column is recirculated to the first diaryl carbonate distillation column.

The side stream column preferably has at least one region. It is particularly preferably operated as a pure enrichment section and preferably has a separating power of from 1 to 50, particularly preferably from 2 to 30 and very particularly preferably from 5 to 20, theoretical plates.

The side stream column is operated at a pressure at the top of from 1 to 1000 mbar (absolute), particularly preferably from 1 to 100 mbar (absolute) and very particularly preferably from 5 to 50 mbar (absolute), and preferably at a reflux ratio of from 0.1 to 10, particularly preferably from 0.2 to 5 and very particularly preferably from 0.2 to 2.

The condensation of the vapours at the top of the side stream column can be effected in one or more stages in an overhead condenser. It is preferably carried out in one or two stages in a temperature range from 70 to 250° C., particularly preferably from 90 to 230° C. and very particularly preferably from 90 to 210° C. The heat obtained in the condensation can preferably be used for generating heating steam or for heating other process sections, e.g. process sections in the preparation of diaryl carbonates. The condensate obtained in the condensation is partly returned as runback to the side stream column. The remaining part of the condensate is taken off as distillate (purified diaryl carbonate). Inert and/or uncondensed vapours are discharged.

In the case of the particularly preferred variant in which the second diaryl carbonate distillation column does not have a stripping section, the enrichment section of this second diaryl carbonate distillation column can be integrated into the first diaryl carbonate distillation column. Here, part of the vapours coming from the lower stripping section of the first distillation column goes into an integrated enrichment section in order to reduce the content of high boilers. The vapours leaving the top of this integrated side stream column are condensed in the external condenser(s) and partly returned as runback to the top of the second diaryl carbonate distillation column. The remaining part of the condensate is taken off as distillate (purified diaryl carbonate). Uncondensed vapours are discharged.

In a further particularly preferred variant of the particularly preferred embodiment of the process for preparing diaryl carbonate using a second diaryl carbonate distillation column, this second diaryl carbonate distillation column has both at least one enrichment section and at least one stripping section.

The second diaryl carbonate distillation column has both a stripping section and an enrichment section. The gaseous side stream from the first diaryl carbonate distillation column can firstly be condensed in a single-stage or multistage side stream condenser and subsequently be fed to the second diaryl carbonate distillation column. The second diaryl carbonate distillation column is preferably operated at a pressure at the top of from 1 to 1000 mbar (absolute), preferably from 1 to 100 mbar (absolute) and particularly preferably from 5 to 50 mbar (absolute). Here, the temperature at the bottom is from 150 to 300° C., preferably from 160 to 240° C. and particularly preferably from 180 to 230° C.

The second diaryl carbonate distillation column preferably has a total separation power of from 5 to 100 theoretical plates, preferably from 10 to 80 theoretical plates, particularly preferably from 30 to 80 theoretical plates, with the enrichment section thereof having a separating power of from 1 to 99, preferably from 1 to 79 and particularly preferably from 2 to 79. This column is preferably operated at a reflux ratio of from 0.5 to 20, preferably from 1 to 10 and particularly preferably from 1 to 5.

The condensation of the vapours at the top of the second diaryl carbonate distillation column can be effected in one or more stages in an overhead condenser. It is preferably carried out in one or two stages in a temperature range from 70 to 250° C., particularly preferably from 90 to 230° C. and very particularly preferably from 90 to 210° C. The heat obtained in the condensation can preferably be used for generating heating steam or for heating other process sections, e.g. process sections in the preparation of diaryl carbonates. The condensate obtained in the condensation is partly returned as runback to the second diaryl carbonate distillation column. The remaining part of the condensate is taken off as distillate (purified diaryl carbonate). Uncondensed vapours are discharged.

The vaporization of the liquid running down from the stripping section of the second diaryl carbonate distillation column can likewise be effected in one or more stages in a vaporizer.

The bottom product from the second diaryl carbonate distillation column can subsequently be entirely or partly discharged from the process and/or entirely or partly recirculated to the first diaryl carbonate distillation column.

The above-described particularly preferred embodiment of the process using a second diaryl carbonate distillation column is particularly suitable for the purification of diaryl carbonates having increased requirements in terms of quality. Such increased requirements can be, for example, a reduced proportion of high-boiling secondary components, and the proportion thereof in the diaryl carbonate can be reduced by from 10 to 100% by weight, preferably from 20 to 90% by weight and particularly preferably from 25 to 80% by weight, compared to the process having only one distillation column.

In preferred embodiments, at least one of the reaction columns used in the process and/or at least one of the distillation columns used in the process can have one or more overhead condensers which are integrated into the column, with the ratio d/D of diameter of the steam line from the column to the overhead condenser(s) (d) to the column diameter of the column (D) being in the range from 0.2 to 1, preferably in the range from 0.5 to 1. In a particularly preferred embodiment, the overhead condenser can be integrated into the distillation column so that no steam line between distillation column and overhead condenser is necessary. The ratio d/D is in this case 1. The column cross section after entry into the overhead condenser may also be matched to the progress of the condensation. Corresponding arrangements are also possible for the other distillation columns and/or reaction columns used in the process. Preference is given to a plurality of the reaction columns and/or distillation columns used in the process having one of the abovementioned overhead condensers.

For some forms of condenser, it can be advantageous to make the column cross section variable. If, for example, the vapours to be condensed are conveyed from the bottom upward, the amount of vapour decreases in the upward direction. Reducing the column diameter in the direction of the top of the column matches the column cross section available to the vapour to the decreasing amount of vapour in the upwards direction. The uncondensed vapours do not necessarily have to be taken off at the top. If, for example, a construction in which a bundle of plates or tubes is inserted from the top into the column is selected, the uncondensed vapours can also be taken off at the side.

In preferred embodiments, lines and apparatuses which convey mixtures having a solidification point above 30° C., preferably above 40° C., can be heated to temperatures above this solidification point, preferably to temperatures of more than 1° C. above this solidification point, particularly preferably to temperatures of more than 5° C. above this solidification point. In this way, precipitation of solids within these lines and apparatuses is avoided and restarting of the corresponding plants after downtimes is made considerably easier.

In particularly preferred embodiments, the energy of condensation obtained in one or more condenser(s) selected from the group consisting of
i. the optional intermediate condenser(s) of the first reaction column(s) (diaryl carbonate preparation),
ii. the condenser(s), preferably overhead condenser(s), of the second or further reaction column(s) (diaryl carbonate preparation),
iii. the condenser(s) for condensing the side stream containing the purified diaryl carbonate or, in the case of a second diaryl carbonate distillation column or side stream column being present, preferably the overhead condenser(s) of the second diaryl carbonate distillation column or the side stream column, preferably the side stream column, and
iv. the condenser(s) for condensing the gaseous side stream from the second intermediate boiler column (diaryl carbonate preparation),
   is conveyed directly or indirectly, entirely or partly to the process stage for purifying the dialkyl carbonate in the process for preparing the dialkyl carbonate and is preferably used for intermediate heating of the internal liquid stream in the distillation column for purifying the dialkyl carbonate.

The energy of condensation liberated in the condensers mentioned under iii. and/or iv. is very particularly preferably used for intermediate heating of the internal liquid stream in the column.

For the purposes of this description, direct recirculation of the heat of condensation to the process means that this heat of condensation is returned to the process without an intermediate heating medium, e.g. either for heating one or more streams or for heating one or more column sections within the process. This can, for example, occur in a heat exchanger. Such a heat exchanger is preferably combined with the condenser(s). For the purposes of this description, indirect recirculation of the heat of condensation to the process means that a heating medium which serves for recirculation of the heat of condensation to the process is firstly generated by means of the heat of condensation obtained. This heating medium can, for example, be used for heating one or more streams or one or more column sections within the process. Possible heating media are gases, vapours or liquids, preferably gaseous or liquid industrial heat transfer media such as water, heat transfer media based on mineral oil or synthetic heat transfer media (e.g. Diphyl™, Marlotherm®). Particularly preferred heating media are water and steam.

As a result of the utilization of the heat of condensation, the separation of the alkyl alcohol from dialkyl carbonate can be carried out with a significantly reduced energy consumption. The cooling power in the process stage for preparing diaryl carbonate can be reduced to the same extent. A substantial advantage of the processes described herein over the processes of the prior art is therefore the significant reduction in the energy consumption in the preparation of dialkyl carbonates and diaryl carbonates or alkyl aryl carbonates. At the same time, the process can be carried out using simple apparatus. In the figures, the reference symbols have the following meanings:
K1 Transesterification column
K2 First distillation column for separating the mixture containing dialkyl carbonate and alkyl alcohol
K3 Second distillation column for separating the mixture containing dialkyl carbonate and alkyl alcohol
1 Feed stream containing alkylene carbonate and/or optional catalyst
2 Feed stream containing virtually pure alkyl alcohol
3 Feed stream containing alkyl alcohol and dialkyl carbonate
4 Stream containing alkylene glycol
5 Stream containing purified dialkyl carbonate
6 Stream containing dialkyl carbonate and alkyl alcohol
7 Stream containing virtually pure alkyl alcohol
8 Stream containing extractant (preferably alkylene carbonate)
9 Stream containing extractant (preferably alkylene carbonate)
10 Stream containing extractant (preferably alkylene carbonate)
K11 First reaction column (diaryl carbonate preparation)
K12 Second reaction column (diaryl carbonate preparation)
K13 First distillation column for purifying diaryl carbonate (diaryl carbonate preparation)
K14 Side stream column for purifying diaryl carbonate
K17 First intermediate boiler column for separating off compounds which have a boiling point lower than that of the aromatic hydroxy compound, at the top of the column
K18 Second intermediate boiler column for separating off, inter alia, the aromatic hydroxy compound in the side stream
i. Intermediate condenser for the first reaction column (K11) of the process for preparing diaryl carbonate
ii. Overhead condenser of the second reaction column (K12) of the process for preparing diaryl carbonate
iii. Overhead condenser of the side stream column (K14) or condenser in the side stream of the first distillation column (K13) for purifying the diaryl carbonate in the process for preparing diaryl carbonate
iv. Condenser for the side stream of the second intermediate boiler column (K18) of the process for preparing diaryl carbonate

### DESCRIPTION OF THE DRAWINGS

FIG. 1 describes a step for the transesterification of alkylene carbonate and alkyl alcohol by means of reactive rectification in a first transesterification column (K1) in general and the work-up of the mixture containing dialkyl carbonate and alkyl alcohol which is obtained at the top of the transesterification column by means of dual pressure distillation in a first distillation column (K2) and a second distillation column (K3) with an intermediate heater (a) in the first distillation column.

FIG. 2 describes a step for the transesterification of alkylene carbonate and alkyl alcohol by means of reactive rectification in a first transesterification column (K1) in general and the work-up of the mixture containing dialkyl carbonate and alkyl alcohol which is obtained at the top of the transesterification column by means of a single distillation column containing an intermediate heater (a).

FIG. 3 describes a step for the transesterification of alkylene carbonate and alkyl alcohol by means of reactive rectification in a first transesterification column (K1) in general and the work-up of the mixture containing dialkyl carbonate and alkyl alcohol which is obtained at the top of the transesterification column by means of extractive distillation in a first distillation column (K2) and a second distillation column (K3) with an intermediate heater (a) in the first distillation column and optionally an intermediate heater (a′) in the second distillation column, with the alkylene carbonate preferably being used as extractant.

FIG. 4 describes a step for the transesterification of alkylene carbonate and alkyl alcohol by means of reactive rectification in a first transesterification column (K1) in general and the work-up of the mixture containing dialkyl carbonate and alkyl alcohol which is obtained at the top of the transesterification column by means of distillation and vapour permeation in a distillation column (K2) having an intermediate heater (a).

FIG. 5 describes a step for the transesterification of alkylene carbonate and alkyl alcohol by means of reactive rectification in a first transesterification column (K1) in general and the work-up of the mixture containing dialkyl carbonate and alkyl alcohol which is obtained at the top of the transesterification column by means of distillation and pervaporation in a distillation column (K2) having an intermediate heater (a).

FIG. 6 describes in general terms part of the process for preparing diaryl carbonate from dialkyl carbonate and an aromatic monohydroxy compound by means of reactive rectification in a first reaction column (K11), a second reaction column (K12), a distillation column (K13) for purifying the crude diaryl carbonate, a side stream column (K14) connected to these distillation columns for further purifying the diaryl carbonate, a first intermediate boiler column (K17) and a second intermediate boiler column (K18), where i., ii., iii. and iv. denote the heat exchangers in which the energy of condensation can be recovered and by means of which the intermediate heater(s) (a and/or a′) in the distillation columns K2 and/or K3 of the process stage for preparing dialkyl carbonate is/are operated.

FIG. 7 describes the purification of the diaryl carbonate by means of a distillation column and the offtake and condensation of the diaryl carbonate in the side stream by means of the condenser iii., where the heat of condensation is used for the intermediate heater(s) (a and/or a′) in the distillation columns K2 and/or K3 of the process stage for preparing dialkyl carbonate.

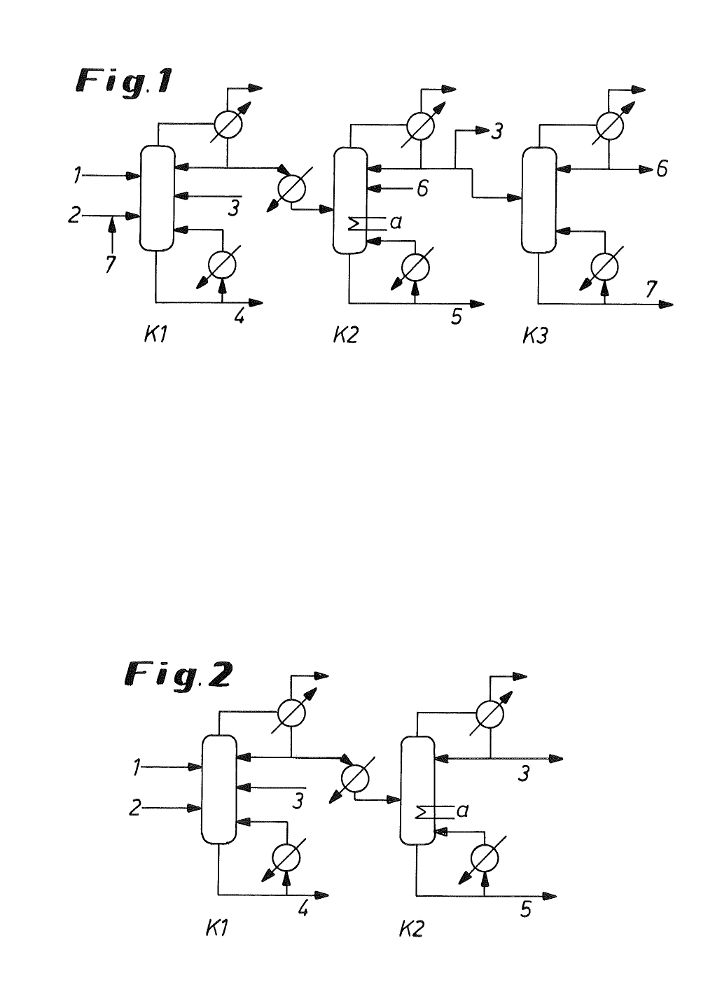

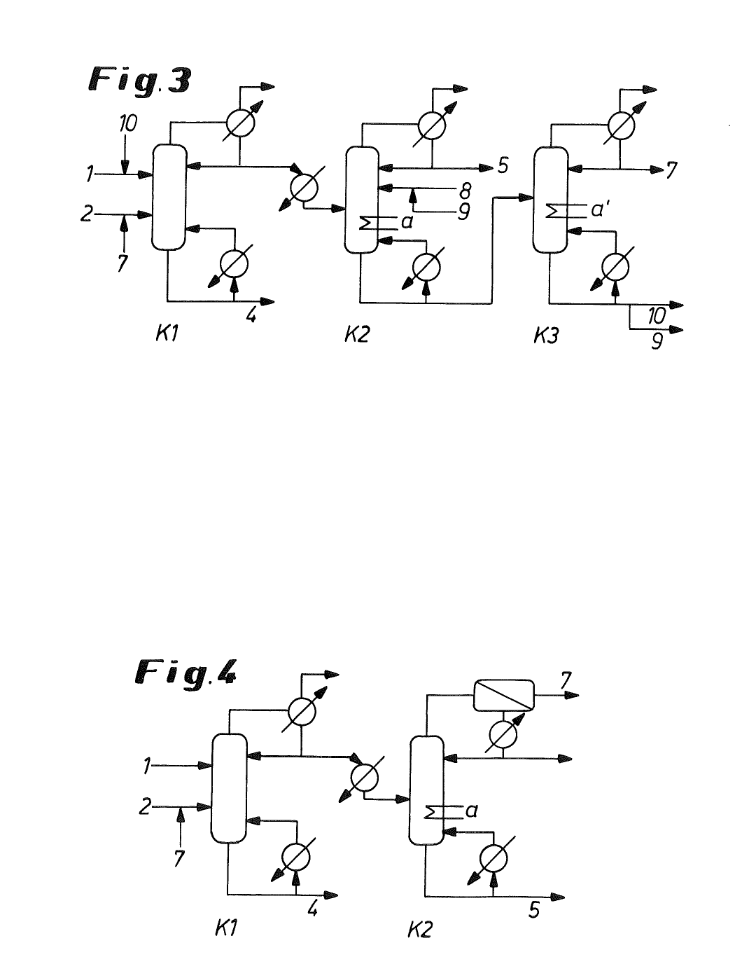

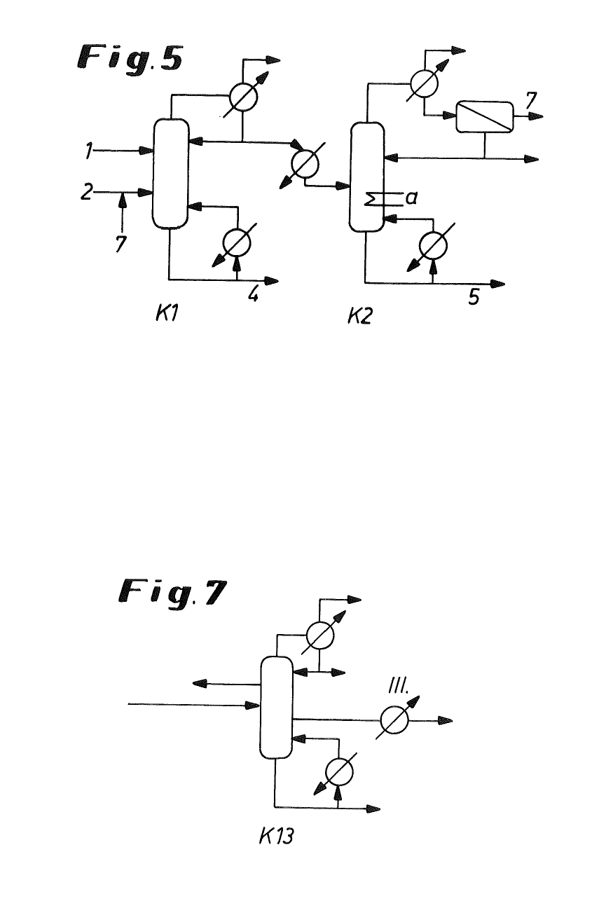

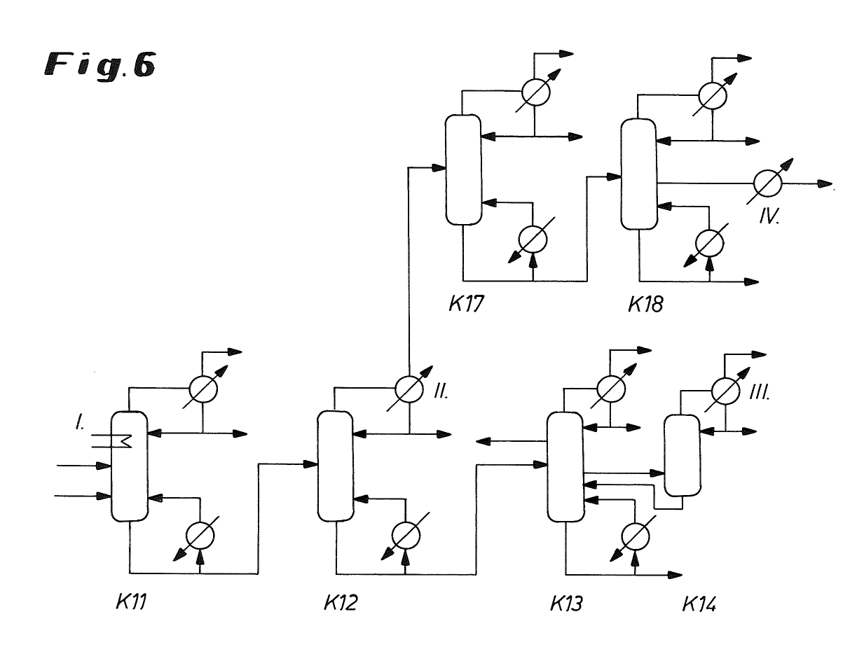

The preferred mode of operation for the process will now be described in detail with the aid of an example. Example 1 shows the preferred mode of operation for the dialkyl carbonate purification column. This example should in no way be interpreted as limiting the invention.

The advantage of the processes described herein, namely the reduction in the consumption of heat energy at the temperature level T_{BV}, which is preferably made available in the form of heating steam, by installation of a technical apparatus for intermediate heating, over other modes of operation without said intermediate heater is demonstrated in the examples.

### EXAMPLE

A distillation column for purifying the dialkyl carbonate formed in the transesterification, which comprises an enrichment section having 28 theoretical plates and a stripping section having 11 theoretical plates, is operated at a pressure measured at the top of the column of 10 bar (absolute) and a reflux ratio of 1.0.

In the lower region of the column, 30 644 kg/h of a dialkyl carbonate-containing alcohol mixture containing 59% by weight of MeOH and 41% by weight of dimethyl carbonate is fed in continuously between the 27th and 28th theoretical plate.

A partial condenser condenses the vapour stream at the top of the column at 137° C. This gives both 21 kg/h of gaseous distillate and 21 378 kg/h of liquid distillate having a composition of 84% by weight of methanol and 16% by weight of dimethyl carbonate.

An apparatus for intermediate heating having a heating power of 6000 kW is installed on the 28th theoretical plate of the column and is operated by means of steam at a pressure of 6 bar as heating medium. The heating steam mentioned is obtained from the subsequent process chain, namely from the preparation of diphenyl carbonate.

9245 kg/h of liquid bottom product having a composition of 99.5% by weight of dimethyl carbonate and 0.5% by weight of methanol are obtained. The bottom vaporizer is operated by means of heating steam at a pressure of 16 bar at 183° C. and has a heating power of 4391 kW.

### COMPARATIVE EXAMPLE

The same conventional distillation column as described in the Example is used for purifying the dialkyl carbonate formed in the transesterification. The column is operated at a pressure measured at the top of the column of 10 bar (absolute) and a reflux ratio of 1.0.

30 644 kg/h of a dialkyl carbonate-containing alcohol mixture containing 59% by weight of MeOH and 41% by weight of dimethyl carbonate are fed continuously into the upper region of the column directly above the first reaction plate.

A partial condenser condenses the vapour stream at the top of the column at 137° C. This gives both 21 kg/h of gaseous distillate and 21 378 kg/h of liquid distillate having a composition of 84% by weight of methanol and 16% by weight of dimethyl carbonate.

9245 kg/h of liquid bottom product having a composition of 99.5% by weight of dimethyl carbonate and 0.5% by weight of methanol are obtained. The bottom vaporizer is operated by means of heating steam at a pressure of 16 bar at 183° C. and has a heating power of 10 396 kW.

Thus, a process for purifying dialkyl carbonates is disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A process for purifying dialkyl carbonates in at least one distillation column containing at least one enrichment section in the upper part of the column and at least one stripping section in the lower part of the column, the process comprising:
heating the internal liquid stream using a technical apparatus in the distillation column, which is used for working up the dialkyl carbonate/alkyl alcohol mixture taken off at the top of a transesterification column; and
recovering, partly or entirely, the energy used to heat the internal liquid stream in the distillation column from another chemical production process.

2. The process according to claim 1, wherein the energy obtained as heat of condensation by condensation in the technical apparatus is fed entirely or partly, directly or indirectly into the process for heating the internal liquid stream in the column.

3. The process according to claim 1, wherein the technical apparatus is positioned in a stripping section of the distillation column.

4. The process according to claim 1, wherein the technical apparatus is positioned outside the distillation column.

5. The process according to claim 1, wherein the technical apparatus is used in an upper half of a stripping section of the distillation column.

6. The process according to claim 1, wherein the energy for heating the internal liquid stream in the column is obtained from a subsequent preparation of diaryl carbonate.

7. The process according to claim 6, wherein the energy from the heat obtained during condensation is obtained from at least one step in the diaryl carbonate preparation selected from among:
an intermediate condenser of a first reaction column of the diaryl carbonate preparation,
a condenser of a second reaction column of the diaryl carbonate preparation,
a condenser for condensing a side stream containing purified diaryl carbonate or, in the case of a second diaryl carbonate distillation column or a side stream column being present, one of the second diaryl carbonate distillation column or the side stream column,
a condenser for condensing a gaseous side stream from a second intermediate boiler column of the diaryl carbonate preparation.
